SEQUENCE LISTING (13 sequences)

SEQ ID NO: 1 - DNA, 22 bp, Artificial
  Adenosine receptor A1 5′ primer
aacattgggccacagacctact

SEQ ID NO: 2 - DNA, 20 bp, Artificial
  Adenosine receptor A1 3′ primer
gcagcacccagacgaagaag

SEQ ID NO: 3 - DNA, 19 bp, Artificial
  Adenosine receptor A2a 5′ primer
cgccatcgaccgctacatc

SEQ ID NO: 4 - DNA, 18 bp, Artificial
  Adenosine receptor A2a 3′ primer
gggccgccagaaaaatcc

SEQ ID NO: 5 - DNA, 21 bp, Artificial
  Adenosine receptor A2b 5′ primer
gacgtggctgtgggactcttc

SEQ ID NO: 6 - DNA, 22 bp, Artificial
  Adenosine receptor A2b 3′ primer
cacagggcagcagctcttattc

SEQ ID NO: 7 - DNA, 22 bp, Artificial
  Adenosine receptor A3 5′ primer
aagccaacaataccacgacgag

SEQ ID NO: 8 - DNA, 20 bp, Artificial
  Adenosine receptor A3 3′ primer
agcagaggcccaggaatagc

SEQ ID NO: 9 - DNA, 21 bp, Artificial
  RhoA sequence primer
tccgtctttggtctttgctga

SEQ ID NO: 10 - DNA, 21 bp, Artificial
  RGC32 siRNA1 sense
gugcagacucguuguacagtt

SEQ ID NO: 11 - DNA, 21 bp, Artificial
  RGC32 siRNA1 antisense
cuguacaacgagucugcactc

SEQ ID NO: 12 - DNA, 21 bp, Artificial
  RGC32 siRNA2 sense
ggaguuugagauucuuaugtt

SEQ ID NO: 13 - DNA, 21 bp, Artificial
  RGC32 siRNA2 antisense
cauaagaaucucaaacucctt

(19) United States
(12) Patent Application Publication
(10) Pub. No.: US 2006/0183147 A1
(43) Pub. Date: Aug. 17, 2006

(54) Methods of identifying genes which modulate myelination

(75) Inventor: Anke Meyer-Franke, Menlo Park, CA (US)

Correspondence Address: DRINKER BIDDLE & REATH (DC), 1500 K STREET, N.W., SUITE 1100, WASHINGTON, DC, 20005-1209, US

(73) Assignee: Elan Pharmaceuticals, Inc.

(21) Appl. No.: 11/352,907

(22) Filed: Feb. 13, 2006

Related U.S. Application Data
(60) Provisional application No. 60/652,477, filed on Feb. 11, 2005.

(51) Int. Cl. C12Q 1/68

(52) U.S. Cl. 435/6

(57) ABSTRACT

The present invention relates to methods of identifying and/or characterizing genes that are modulated during myelination or remyelination, as well as in demyelinating diseases, such as in multiple sclerosis. The invention further provides methods of treating diseases related to myelination in a mammal, such as multiple sclerosis, by administering agents that promote remyelination of oligodendrocytes.

### CROSS-REFERENCE TO RELATED APPLICATION

The present application is entitled to priority under 35 U.S.C. § 119(e), to U.S. Provisional Application No. 60/652,477, filed on Feb. 11, 2005, which application is hereby incorporated herein by reference in its entirety.

### REFERENCE TO A SEQUENCE LISTING

This application includes a sequence listing in accordance with 37 C.F.R. 1.821 et seq., which sequence listing is hereby incorporated by reference in its entirety.

### FIELD OF THE INVENTION

The application relates to methods of identifying and/or characterizing genes that are modulated in myelination, remyelination, and in demyelinating diseases, such as in multiple sclerosis. The invention further provides methods of treating multiple sclerosis in a mammal by administering agents that promote remyelination of oligodendrocytes.

### BACKGROUND

Even though there is considerable remyelination and repair following the first immune attacks in multiple sclerosis (MS), there is a loss of ability to remyelinate over time, causing progressive neurodegeneration in patients in the secondary progressive phase of the disease. The loss of ability to remyelinate could be due to either the lack of potential precursor cells of myelinating oligodendrocytes or the failure of oligodendrocytes to myelinate. It has been observed that in the beginning it is a failure of newly formed oligodendrocytes to myelinate, followed by a depletion of progenitor cells over time.

Immunohistochemistry with antibodies against developmental surface markers (Trapp et al., 1998 New England Journal of Medicine 338: 278-285) has shown that the existence of oligodendrocyte precursor cells (OPCs) in the brains of MS patients. OPCs seem to be recruited to the site of lesions, where they differentiate into oligodendrocytes, positive for myelin proteins. These oligodendrocytes adhere to axons and form a few loose loops of membrane around them, but fail to produce internodes of compact myelin. Failing to myelinate, these oligodendrocytes will undergo cell death.

There is not much known about why oligodendrocytes fail to myelinate, since the process of myelination is still not well understood. Oligodendrocytes differentiate from progenitor cells that can still divide to fate-committed oligodendrocytes, which are positive for sulfatide (the 04 antibody epitope) to oligodendrocytes that express myelin proteins such as myelin-basic-protein (MBP). The final step of maturation though is the competence to form internodes of compacted myelin along axons.

In vivo oligodendrocytes are found adjacent to neuronal tracts but do not form myelin until the neurons have innervated the appropriate target and formed functional synapses. This suggests that oligodendrocytes are kept from maturation until they receive an instructive neuronal signal.

These observations are suggestive of electrical activity as a potential signal that controls the down-regulation of inhibitory molecules and causes the up-regulation of myelination promoting factors. Several molecules have been proposed to play a role in myelination, including the polysialic acid linked to the adhesion molecule NCAM, which inhibits the adhesion of oligodendrocytes to axons. The notch—1 receptor ligand “jagged” is expressed on the neuritic side, keeping the oligodendrocyte “immature.” Recently, up-regulation of both molecules was found in the gene expression profiling of acute MS patient lesions. Even though oligodendrocytes are known to have neurotransmitter receptors (mostly in vitro evidence), e.g. for neurotransmitters like glutamate, GABA, serotonin, it has been difficult to identify the link between electrical activity and the regulation of such molecules. Recent work suggests that the release of adenosine from stimulated dorsal root ganglion cell axons induces calcium mobilization in cortical oligodendrocytes (Stevens et al., 2002 Neuron 36:855-868). Adding adenosine or a pan-adenosine receptor agonist to these co-cultures increased the amount of myelin observed, thus linking calcium mobilization due to electrical activity with the competence to myelinate.

A new approach to the identification of targets has been discovered. The optic nerve in rodents is one of the tracts in which in vivo myelination has been studied in the past. In the rat innervation of the optic tectum (superior colliculus) by retinal ganglion cell axons takes place beginning from embryonic day 18 forward. Myelination starts about postnatal day 7 and is complete by postnatal day 14.

In vitro, co-cultures of purified rat retinal ganglion cells with purified rat optic nerve oligodendrocytes present a model system for failed myelination, resembling the phenotype observed in MS lesions. The oligodendrocytes do adhere to axons, but due to the re-expression of “jagged” or other inhibitory molecules on the cultured retinal ganglion cell axons, they do not form compact myelin. Recent work has also shown that contact with target tissue is necessary to trigger myelination in oligodendrocyte cultures, likely due to the formation of functional synapses that will trigger the correct electrical activity (Wang et al., 1998 Neuron 21:63-75). This might lead to down-regulation of molecules, such as “jagged,” by so far unknown signal transduction pathways on the axonal side and to the secretion of factors like adenosine, stimulating the oligodendrocytes to become competent to form compact myelin. It had also been described that electrically active neurons secrete factors such as adenosine that can stimulate a non-myelinating oligodendrocyte to become a myelinating oligodendrocyte (Stevens et al., 2002 Neuron 36:855-868).

The secondary progressive phase of MS is characterized by continuous demyelination and progressive neurodegeneration, likely due to a decrease in the ability to remyelinate. It is hypothesized that chronic demyelination is mediated by macrophages/microglia, continuing to attack myelin. This continuous demyelination leads to more dystrophic neurons that over time cannot be remyelinated anymore, leading to progressive neurodegeneration.

In order to address both aspects of this stage of MS, there is a need to establish a cellular system that would represent the situation in MS lesions. Additionally, a better understanding of the factors regulating myelination and remyelination, and particularly, the role of oligodendrocytes, is required in order to effectively treat diseases involving aberrant myelination or demyelination, such as MS. The present invention addresses and meets these needs.

### SUMMARY OF THE INVENTION

The methods and compositions set forth herein provide a way to identify a gene that is regulated during myelination of an oligodendrocyte. The methods include the step of comparing the gene expression profiles of at least two oligodendrocytes, wherein the oligodendrocytes are differentiated in vitro to the state of myelin protein expression, further wherein a first oligodendrocyte is differentiated in the presence of an adenosine receptor agonist and a second oligodendrocyte is differentiated in the absence of the adenosine receptor agonist. The methods also include the steps of identifying a molecule, the expression of which molecule is altered in response to adenosine receptor agonist treatment, wherein the altered expression of the molecule is an indication that the oligodendrocyte in which said expression is altered is competent to form compact myelin. The methods also include the steps of identifying the gene encoding the identified molecule.

The methods and compositions may also include an oligodendrocyte, which can be selected from the group consisting of an optic nerve oligodendrocyte, a cortical oligodendrocyte, and a spinal cord oligodendrocyte. In one aspect, the oligodendrocytes are essentially pure. In another aspect, the oligodendrocytes are at least 85%, 90%, 95% or 99% pure.

The methods and compositions may also include an identifying step comprising at least one of the methods selected from the group consisting of interfering RNA (RNAi), a lentiviral expression system, an adenoviral expression system, a blocking antibody, and a cell permeable inhibitor.

In an aspect, a molecule identified according to the methods of the invention is a sphingosine kinase.

In another aspect, an adenosine receptor agonist may be selected from the group consisting of adenosine, MECA 9-[5(methylcarbamoyl)-β-D-ribofuranosyl]adenosine, CPA (N⁶-cyclopentadyladenosine), CHA (N⁶-cyclohexyladenosine), CCPA (2-chloro-CPA), NECA (N-ethylcarboxamidoadenosine, and XAC (8-[4-[[[[(2-aminoethyl)amino]carbonyl]methyl]oxy]phenyl]-1,3-dipropylxanthine. In an embodiment, an adenosine receptor agonist is specific for an adenosine A1 receptor. In another embodiment, an adenosine receptor agonist can bind to at least two different types of adenosine receptors.

The methods and compositions herein also provide a cell culture system for identifying a gene that is regulated during oligodendrocyte myelination. A culture system comprises at least two oligodendrocytes, wherein the oligodendrocytes are differentiated in vitro to a state of myelin protein expression and wherein (i) one of said oligodendrocytes is differentiated in the presence of an adenosine receptor agonist; and (ii) the second of said oligodendrocytes is differentiated in the absence of an adenosine receptor agonist. The culture system further comprises an apparatus for comparing the gene expression profiles of the first and second oligodendrocytes to identify genes that modulate myelin formation by an oligodendrocyte. In one aspect of a cell culture system each oligodendrocyte is independently selected from the group consisting of an optic nerve oligodendrocyte, a cortical oligodendrocyte, and a spinal cord oligodendrocyte.

In an embodiment, the expression of a molecule is increased. In another embodiment, the expression of a molecule is decreased.

In an embodiment, the process of myelination is remyelination. In another embodiment, myelination comprises remyelination and new myelination. In yet another embodiment, the myelination state of at least one of said oligodendrocytes is confirmed by direct detection of myelination.

In an embodiment of the methods and compositions set forth herein, the myelination state of at least one oligodendrocyte is confirmed by detection of a surrogate marker for myelination. In an aspect, the surrogate marker is myelin oligodendrocyte glycoprotein (MOG).

In an aspect of the methods and compositions set forth herein, a method of remyelinating an oligodendrocyte comprises modulating sphingosine kinase activity. In another aspect, a method of myelinating an oligodendrocyte comprises modulating sphingosine kinase activity. In one embodiment, sphingosine kinase activity is enhanced. In another embodiment, sphingosine kinase activity is enhanced by way of a sphingosine kinase agonist. In yet another embodiment, sphingosine kinase activity is inhibited. In one aspect, sphingosine kinase activity is inhibited with a sphingosine kinase antagonist.

The methods and compositions herein also provide a method for treating multiple sclerosis in a mammal, the method comprising administering to a mammal a sphingosine kinase modulator. The methods and compositions herein also provide a method for inhibiting progression of multiple sclerosis in a mammal, the method comprising administering to a mammal a sphingosine kinase modulator. The methods and compositions herein also provide a method for alleviating multiple sclerosis in a mammal, the method comprising administering to a mammal a sphingosine kinase modulator. The methods and compositions herein also provide a method for remyelinating cells in a mammal, the method comprising modulating sphingosine kinase activity in a mammal.

### BRIEF DESCRIPTION OF THE DRAWINGS

It is to be understood that the following detailed description is exemplary and explanatory only, and are not restrictive of the material methods, devices, and kits. The accompanying drawings, which are incorporated herein by reference, and which constitute a part of this specification, illustrate certain embodiments, and together with the detailed description, and serve to explain the principles of the materials and methods. The drawings are exemplary only, and should not be construed as limiting the materials, methods, and compositions described herein.

FIG. 1 is a graph depicting the results of quantitative RT-PCR demonstrating adenosine receptor expression in early-stage (“young”) and late-stage (“old”) oligodendrocyte differentiation.

FIG. 2, comprising FIGS. 2A through 2E, is a series of images depicting purified rat optic nerve oligodendrocytes grown in proliferation-promoting medium, then exchanged for differentiation-promoting medium and then fixed for immunohistochemistry on the indicated days. Cells were immunolabeled with the oligodendrocyte-specific antibody against sulfatide, O4.

FIG. 3, comprising FIGS. 3A through 3E, is a series of images from the cells treated as described in FIG. 2, and then immunolabeled with antibodies against galactocerebroside (O1) and MBP. For quantification, approximately 200 cells were counted per well.

FIG. 4, comprising FIGS. 4A through 4D, is a series of images depicting NCAM immunostaining demonstrating knockdown of NCAM expression in oligodendrocytes treated with siNCAM-1 post-transfection. FIGS. 4C and 4D depicts DAPI staining of cell nuclei demonstrating no difference in cell number.

FIG. 5 is a graph illustrating the results of quantitative RT-PCR demonstrating inhibition of NCAM expression in siNCAM treated oligodendrocytes post-transfection.

FIG. 6, comprising FIGS. 6A through 6F, is a series of images depicting optic nerve myelin cultures. FIGS. 6A through 6C depict optic nerve myelin cultures labeled with an anti-MBP antibody (oligodendrocytes). FIGS. 6D through 6F depict optic nerve myelin cultures double labeled with anti-MBP and anti-neurofilament antibody (RGCs). FIGS. 6A and 6D illustrate control cultures with non-myelinating oligodendrocytes, FIGS. 6B and 6E depict myelinating oligodendrocytes that had been stimulated with MECA for in isolation before the co-culture with RGCs. FIGS. 6C and 6F depict co-cultures containing the same myelinating oligodendrocytes as in FIGS. 6B and 6D, but the oligodendroctyes in FIGS. 6C and 6F were treated with MECA continuously for two weeks.

FIG. 7 is a table depicting that the ADORA1 agonist MECA needs to be present for 72 hours to obtain a majority of myelinating oligodendrocytes.

FIG. 8, comprising FIGS. 8A through 8F, is a series of images depicting optic nerve myelination cultures. FIGS. 8A and 8D depict oligodendrocytes visualized with an anti-MBP antibody, FIGS. 8B and 8E depict neurons with an anti-neurofilament antibody. FIGS. 8C and 8F illustrate a double-label for both antibodies depicted in FIGS. 8A and 8B. FIGS. 8A through 8C depict control cultures with un-stimulated oligodendrocytes in co-culture with RGCs, FIGS. 8D through 8F depict MECA-stimulated oligodendrocytes.

FIG. 9, comprising FIGS. 9A-1 through 9C-6, is a series of images depicting oligodendrocyte labeling. FIGS. 9A-1 through 9A-3, 9B-1 through 9B-3, and 9C-1 through 9C-3 illustrate control cells. FIGS. 9A-4 through 9A-6, 9B-4 through 9B-6, and 9C-4 through 9C-6 illustrate MECA-stimulated cultures. FIG. 9A depicts oligodendrocyte membrane labeled with an anti-MBP antibody, FIG. 9B depicts RGC neurons labeled with an anti-neurofilament antibody, and FIG. 9C depicts a double-label with both antibodies.

FIG. 10 is a graph illustrating the results of quantitative RT-PCR demonstrating RhoA expression in MECA treated oligodendrocytes.

FIG. 11, comprising FIGS. 11A and 11B, illustrate the effect of siRNA on the cell cycle protein RGC32. FIG. 11A is a series of images depicting the MOG surface staining with a MOG-specific antibody, illustrating that MOG expression is reduced when RGC32 is inhibited. FIG. 11B is a graph depicting a decrease in MOG expression when RGC32 is inhibited, as determined by QRT-PCR.

FIG. 12, comprising FIGS. 12A through 12F, is a series of scatter plots comparing gene expression intensities within replicates of control and MECA treated cortical oligodendrocytes.

FIG. 13, comprising FIGS. 13A through 13C, is a series of scatter plots demonstrating distribution of genes between control and MECA treated cortical oligodendrocytes.

FIG. 14, comprising FIGS. 14A through 14D, illustrates a comparison of differentially regulated genes grouped into functional classes. FIG. 14A illustrates a table listing differentially regulated genes grouped into functional classes from ON-D 1 with p-value<0.05 and a 1.5 fold cutoff showing genes in common with ON-D3 and cortical d1. FIG. 14B is a table illustrating differentially regulated genes grouped into functional classes from ON-D1 with p-value<0.05 and a 1.5 fold cutoff showing only genes in common with ON-D3 and cortical d1. Genes exclusive to ON-D1 have been removed from the listing set forth in FIG. 14B. FIG. 14C is a table illustrating clustering based on similarities in expression profiles of differentially regulated genes (p-value<0.05, 1.2 fold-change cutoff for ON-D 1) common to ON-D 1, ON-D2 and cortical d1 oligodendrocytes. Exemplars for each of the clusters are underlined and shown as scatter plots. FIG. 14D is a table illustrating clustering based on similarities in expression profiles of differentially regulated genes (p-value<0.05, 1.2 fold-change cutoff for ON-D1) common to ON-D1 and cortical d1 oligodendrocytes. Exemplars for each of the clusters are underlined and shown as scatter plots.

FIG. 15 is a table confirming genes that were identified on an array by QRT-PCR.

FIG. 16, comprising FIGS. 16A through 16L, is a series of images depicting labeled oligodendrocytes. FIGS. 16A through 16F depict control optic nerve oligodendrocytes, FIGS. 16A through 16C depict oligodendrocytes labeled with anti-MOG antibody, FIGS. 16D through 16F depict the nuclei labeled with DAPI. FIGS. 16G through 16L depict MECA-stimulated oligodendrocytes. FIGS. 16G through 161 depict oligodendrocytes labeled with anti-MOG antibody and FIGS. 16J through 16L depict oligodendrocytes labeled with DAPI.

FIG. 17, comprising FIGS. 17A through 17L, is a series of images depicting labeled oligodendrocytes. FIGS. 17A through 17F depict cortical-enriched oligodendrocytes, FIGS. 17A through 17C depict oligodendrocytes labeled with anti-MOG antibody, FIGS. 17D through 17F depict the nuclei labeled with DAPI. FIGS. 17G through 17L depict MECA-stimulated oligodendrocytes. FIGS. 17G through 171 depict oligodendrocytes labeled with anti-MOG antibody and FIGS. 17J through 17L depict oligodendrocytes labeled with DAPI.

FIG. 18, comprising FIGS. 18A through 18L, is a series of images depicting labeled oligodendrocytes. FIGS. 18A through 18C and FIGS. 18G through 18I depict cortical oligodendrocytes, control and MECA-stimulated for different time points, labeled with anti-MOG antibody. FIGS. 18D through 18F and FIGS. 18J through 18L depict the nuclei of the same cells depicted in FIGS. 18A-18C and FIGS. 18G-18I labeled with DAPI.

### DETAILED DESCRIPTION

1. Definitions and Acronyms

1.1 Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

It must be noted that as used herein, the singular forms “a”, “an”, and “the” include plural referents unless the context clearly dictates otherwise. Thus, for example, reference to “a patient” includes a plurality of patients, and so forth.

By “patient” or “subject” is meant to include any vertebrate, such as a mammal, amphibian, avian, or ichthyes. Mammals include, but are not limited to, bovines, primates, equines, porcines, caprines, ovines, felines, canines, and any rodent (e.g., rats, mice, hamsters, and guinea pigs). A preferred primate is a human. However, subjects may also be agricultural animals (e.g., chickens and other fowl) and domesticated animals.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence. The term “nucleic acid” typically refers to large polynucleotides.

A “polynucleotide” means a single strand or parallel and anti-parallel strands of a nucleic acid. Thus, a polynucleotide may be either a single-stranded or a double-stranded nucleic acid. The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins from other species (homologs), which have a nucleotide sequence which differs from that of the human proteins described herein are within the present scope. Nucleic acid molecules corresponding to natural allelic variants and homologs of a cDNA can be isolated based on their identity to human nucleic acid molecules using the human cDNAs, or a portion thereof, as a hybridization probe according to standard hybridization techniques under stringent hybridization conditions.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

“Mutants,” “derivatives,” and “variants” of a polypeptide (or of the DNA encoding the same) are polypeptides which may be altered in one or more amino acids (or in one or more base pairs) such that the peptide (or nucleic acid) is not identical to the sequences recited herein, but has the same property as the wild type polypeptide. A “variant” or “allelic or species variant” of a protein or nucleic acid is meant to refer to a molecule substantially similar in structure and biological activity to either the protein or nucleic acid. Thus, provided that two molecules possess a common activity and may substitute for each other, such as playing a role in myelination, they are considered variants as that term is used herein even if the composition or secondary, tertiary, or quaternary structure of one of the molecules is not identical to that found in the other, or if the amino acid or nucleotide sequence is not identical.

A “recombinant polypeptide” is one which is produced upon expression of a recombinant polynucleotide.

By the term “specifically binds,” as used herein, is meant a compound, e.g., a protein, a nucleic acid, an antibody, and the like, which recognizes and binds a specific molecule, but does not substantially recognize or bind other molecules in a sample.

As used herein, to “treat” means reducing the frequency with which symptoms of a disease, disorder, or adverse condition, and the like, are experienced by a patient. For example, treating myelination a disorder can mean preventing demyelination or inducing remyelination.

As the term is used herein, “regulation” of the expression of a gene, or of a polypeptide from a gene, refers to any alteration of the expression from between two measurable states. For example, regulation of the expression of a polypeptide may be an increase in the expression of the polypeptide. Alternatively, regulation of the expression of a polypeptide may be a decrease in the expression of the polypeptide.

As the term is used herein, “population” refers to two or more cells.

“Substantially pure,” as the term is used herein, refers to a population of a substance that that is comprised of that substance between a level of greater than 50% to a level of up to 90%. For example, in a population of 100 various cells, an oligodendrocyte can be considered to be “substantially pure” if the population comprises at least 51 oligodendrocytes. Similarly, an oligodendrocyte can be considered to be “substantially pure” if the a population of glial cells comprises about 80% oligodendrocytes.

“Essentially pure,” as the term is used herein, refers to a population of a substance that is comprised primarily of that substance, from a level of about 90% to a level of about 100%. A cell population comprising 90.1% oligodendrocytes is “essentially pure” as the term is used herein. Similarly, a cell population comprising 99.9% oligodendrocytes is “essentially pure” as the term is used herein. Purity is assessed herein with respect to the separation of oligodendrocytes from other types of cells. In one aspect, purity is assessed with respect to the separation of oligodendrocytes from other glial cells.

A “defined culture medium” as the term is used herein refers to a cell culture medium with a known composition.

A molecule (e.g., a ligand, a receptor, an antibody, and the like) “specifically binds with” or “is specifically immunoreactive with” another molecule where it binds preferentially with the compound and does not bind in a significant amount to other compounds present in the sample. A “ligand” is a compound that specifically binds with a target receptor. A “receptor” is a compound that specifically binds to a ligand.

By “immunoglobulins” is meant to include antibodies and antibody fragments. As used herein, the term “antibody” is meant to refer to complete, intact antibodies, diabodies, and antibody fragments such as Fab fragments, Fab′, and F(ab)₂ fragments. Complete antibodies include monoclonal antibodies (mAb), such as murine monoclonal antibodies, chimeric antibodies, humanized antibodies, primatized antibodies, and human antibodies. The production of antibodies and genetically engineered or enzymatically produces portions of antibodies and the organization of the genetic sequences that encode such molecules are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); and Breitling et al., RECOMBINANT ANTIBODIES (Wiley-Spektrum, 1999), which are incorporated herein by reference for all purposes. Immunoglobulins also include fragments such as scFv.

By “immunologically active” is meant any immunoglobulin protein or fragment thereof which recognizes and binds to an antigen. Preferably, the immunologically active protein or fragment thereof modulates the antigen to which it binds. For example, if it binds to an oligodendrocyte antigen, the immunologically active protein or fragment thereof would modulate the oligodendrocyte antigen activity.

As used herein, the term “modulate” refers to the act of effecting an alteration or change. For example, the myelination of an oligodendrocyte can be modulated by a compound that stimulates myelination of an oligodendrocyte. Alternatively, the myelination of an oligodendrocyte can be modulated by a compound that decreases myelination of an oligodendrocyte.

As used herein, to “alleviate” a disease, disorder or condition means reducing the severity of one or more symptoms of the disease, disorder or condition.

The term “myelination” is meant to include the production of myelin by an oligodendrocyte, also referred to herein as the expression of myelin by an oligodendrocyte. The term “myelination” may encompass other events and/or processes, but does not necessarily encompass other events and/or processes. Such events and/or processes include, but are not limited to, the maturation of a progenitor oligodendrocyte to a mature, myelinating oligodendrocyte, the outgrowth of myelin from an oligodendrocyte, whereby the myelin contacts and surrounds an axon of a neuron, and up- or down-regulation of one or more genes in an oligodendrocyte, wherein such genes are associated with the process of myelination.

A “state of myelin protein expression,” as the term is used herein, refers to the state of an oligodendrocyte in which the oligodendrocyte is actively expressing and/or maintaining myelin protein.

The term “remyelination” refers to the production of myelin by an oligodendrocyte, wherein the oligodendrocyte had previously produced myelin on at least one occasion. “Remyelination” therefore includes the definition of the term “myelination.” However, “myelination” may or may not include the scope of the term “remyelination.” It should be understood that methods and compositions related to “myelination” of an oligodendrocyte may apply equally to “remyelination” of an oligodendrocyte.

1.2 Acronyms

The following acronyms are used in this application and have the associated term, unless indicated otherwise in the specification.

6FAM 6-carboxyfluorescein

ADORA1 adenosine A1 receptor

cAMP cyclic adenosine monophosphate

CNS central nervous system

CNTF ciliary neurotrophic factor

DAPI 4′,6-diamidino-2-phenylindole dilactate

DNA deoxyribonucleic acid

FBS fetal bovine serum

GABA gamma amino butyric acid

GPCR G-protein coupled receptor

MBP myelin basic protein

MECA 5′-N-methylcarboxamidoadenosine

MOG myelin oligodendrocyte glycoprotein

MS multiple Sclerosis

NCAM neural cell adhesion molecule

NT3 neurotrophin 3

OD oligodendrocyte

OD optical density

ON overnight

ON-D1 optic nerve oligodendrocytes-1 day

ON-D3 optic nerve oligodendrocytes-3 day

OPC oligodendrocyte precursor cells

PBS phosphate buffered saline

PCR polymerase chain reaction

PDGF platelet-derived growth factor

PDL poly-D-lysine

PEI polyethylenimine

qPCR quantitative polymerase chain reaction

QRT-PCR quantitative reverse transcriptase polymerase chain reaction

RGC retinal ganglion cell

RNAi interfering RNA

RT-PCR reverse transcriptase polymerase chain reaction

SD Sprague-Dawley

siNCAM small interfering RNA against NCAM

TAMRA carboxytetramethyl rhodamine

2.0 Description

Provided are methods and compositions for the identification of a gene that is regulated during the myelination of an oligodendrocyte. In one particular aspect are provided methods and compositions for the identification of a gene that is regulated during the remyelination of an oligodendrocyte. This is because it has been shown, for the first time herein, that a gene can be specifically identified, wherein the gene encodes a polypeptide that is regulated during the process of remyelination of an oligodendrocyte.

It has been discovered that the use of a co-culture system of purified rat retinal ganglion cells and optic nerve oligodendrocytes is a system which resembles the phenotype observed in MS lesions. That is, the oligodendrocytes loosely wrap membrane around axons, but fail to form internodes of compact myelin. The retinal ganglion cell (RGC) neurons in such cultures are mostly electrically inactive, similar to the dystrophic neurons observed in MS lesions.

Further, it has been discovered that adenosine can be used in the optic nerve co-culture system to obtain myelin segments on individual axons by approximately 90% of the oligodendrocytes.

In one aspect, the myelinating oligodendrocyte provides a myelin sheath around one or more axons of one or more nerve fibers, the myelin sheath provides the insulation required for the proper electrical conductivity of signaling to and from the nerve cell, enables salutatory propagation of the electrical signals as well as necessary survival factors for neurons. As will be understood by the skilled artisan, the myelin is typically wrapped around the axon multiple times to form a tightly-wound insulating sheath. However, it will also be understood that oligodendrocytes exist in perineural areas as well as in the white matter of the brain. Therefore, in perineural areas of the central nervous system, the myelinating properties of an oligodendrocyte may be differentially regulated, such that the oligodendrocyte can surround and/or support the axon of one or more neurons.

In one embodiment, a method is provided for of identifying a gene that is regulated—either by up-regulation or down regulation—during the processes of myelination or remyelination. In one aspect, the method relates to the role of oligodendrocytes in the development and progression of myelinating disorders, such as MS. As described in detail elsewhere herein, the secondary progressive phase of MS is characterized by continuous demyelination and progressive neurodegeneration, due in part to a decrease in the ability to remyelinate. Therefore, the identification of one or more genes that is regulated during the myelination process can provide a means by which to affect the remyelination process. In particular, modulation of a gene that is regulated during the myelination process can stimulate or enhance myelination, or it can inhibit or prevent myelination of the oligodendrocyte.

Methods and materials presented herein can be used with all myelinating disorders, including MS. When only MS is referred to, it should be construed to also include other myelinating disorders, unless specifically limited to MS. Thus, the materials and methods should be understood to apply equally to other myelinating diseases, and in particular, those myelinating diseases involving oligodendrocytes. Other myelinating diseases include, but are not limited to, deep white matter ischemia, effects of aging, infectious and inflammatory disorders (e.g., progressive multifocal leukoencephalopathy, post-infectious encephalitis, HIV-induced encephalitis), radiation injury, effects of chemotherapeutic agents, effects of immunosuppressant therapy, central pontine myelinolysis, hereditary metabolic disorders (e.g., Hurler's disease, Lowe's syndrome, and leukodystrophies), and Alzheimer's disease.

The identification, diagnosis and classification of a disease or disorder of myelination will be understood by one of skill in the art (see, e.g., Valk et al., MAGNETIC RESONANCE OF MYELIN, MYELINATION AND MYELIN DISORDERS. Springer-Verlag, Berlin 1989, pp. 4-21; Glasier et al., 1995, American Journal of Neuroradiology 16:87-96; Pasco et al., 1991, Ped. Radiol. 21:161; and U.S. Pat. No. 6,933,119, each of which is incorporated by reference herein in its entirety). Similarly, methods of detecting diseases or disorders of myelination are encompassed by the present invention, and will be understood to be broadly applicable to other diseases and disorders of myelination set forth in detail herein.

2.1 Methods of Screening

In an embodiment, a method is provided for identifying a gene that is regulated during myelination of an oligodendrocyte. In an embodiment, a method includes comparing the gene expression profiles of at least two oligodendrocytes, wherein the oligodendrocytes are differentiated in vitro to the state of myelin protein expression. The first oligodendrocyte is differentiated in the presence of an adenosine receptor agonist and the second oligodendrocyte is differentiated in the absence of said adenosine receptor agonist. The method further includes identifying a molecule, the expression of which molecule is altered in response to treatment of an oligodendrocyte with an adenosine receptor agonist. The altered expression of such a molecule is an indication that the oligodendrocyte in which the expression is altered is competent to form compact myelin. Identification of the gene encoding such a molecule thereby identifies a gene that is regulated during myelination of an axon by the oligodendrocyte.

In one aspect, the gene is regulated during remyelination of an axon by an oligodendrocyte. In another aspect, the gene is regulated during new myelination of an axon by an oligodendrocyte. As will be understood, based on the disclosure set forth herein, the process of myelination of an oligodendrocyte may depend upon multiple factors, including, but not limited to, the stage of development of the oligodendrocyte, the age of the oligodendrocyte, the environment of the oligodendrocyte, and the presence or absence of external signals affecting the oligodendrocyte.

Therefore, it will be understood that the methods set forth herein are applicable to oligodendrocytes in vivo, ex vivo, and in vitro. Based on the disclosure set forth herein, the skilled artisan will understand how to establish and verify the conditions and/or components required to screen for a gene that is regulated during the myelination of an oligodendrocyte.

In one aspect, an oligodendrocyte is a progenitor cell. In another aspect, an oligodendrocyte is an adult progenitor cell. An adult progenitor cell is a progenitor cell that exists in adult animals, including humans. In yet another aspect, an oligodendrocyte is in the process of differentiation. In another aspect, an oligodendrocyte is a mature oligodendrocyte. In yet another aspect, a mature oligodendrocyte is a myelinating oligodendrocyte.

Any oligodendrocyte can be used with the materials and methods set forth herein, provided that the interpretation of results obtained with such oligodendrocytes used in a method accounts for the growth state of the oligodendrocyte when evaluating the outcome of the method. That is, if a progenitor oligodendrocyte is used in a method, the steps required to advance such an oligodendrocyte to a myelinating stage must be considered. Similarly, if an oligodendrocyte is already at the stage of a myelinating oligodendrocyte, the genetic and phenotypic changes that have occurred in the oligodendrocyte must be considered when evaluating the outcome of a method.

Oligodendrocytes useful in the materials and methods herein include an optic nerve oligodendrocyte, a cortical oligodendrocyte, and a spinal cord oligodendrocyte, among others. However, the present materials and methods should not be construed to be limited to such oligodendrocytes. It will be understood that any oligodendrocyte capable of myelination, as well as any oligodendrocyte capable of being induced to myelinate, is encompassed herein. This is because the materials and methods set forth herein include methods of screening for genes that are up- or down-regulated during the process of myelination in an oligodendrocyte.

In an embodiment, an oligodendrocyte is substantially pure. In another embodiment, an oligodendrocyte or an oligodendrocyte population is essentially pure. Such an oligodendrocyte or an oligodendrocyte population is one that is largely free of any non-desired components, and the oligodendrocyte is substantially separated from undesired components. Such components may include, but are not limited to, oligodendrocyte cells of another type (e.g., optic nerve oligodendrocytes versus cortical oligodendrocytes), non-oligodendrocyte cells (e.g., non-oligodendrocyte glial cells or neuronal cells), and non-cellular components (e.g., proteins or nucleic acids).

In another embodiment, an oligodendrocyte is at least 99% pure. That is, the oligodendrocyte has been purified away from any non-oligodendrocyte cells, such that the population of the oligodendrocyte comprises at least 99% of the cells in such a population. In one aspect, a pure oligodendrocyte is one that does not contain any oligodendrocytes of a different type. By way of a non-limiting example, a pure optic nerve oligodendrocyte may be free of any non-optic nerve (e.g., cortical) oligodendrocytes. However, a pure oligodendrocyte population may also be one that is free of any non-oligodendrocyte cells. By way of another non-limiting example, a pure oligodendrocyte population may include both optic nerve and cortical oligodendrocytes, but may be free of any neuronal cells or of any non-oligodendrocyte glial cells. As will be understood by the skilled artisan, the nature and extent of the purity of an oligodendrocyte or an oligodendrocyte population may be selected based on the nature of the method.

In yet another embodiment, an oligodendrocyte or an oligodendrocyte population is from about 98% pure to about 99% pure, from about 95% pure to about 98% pure, from about 90% pure to about 95% pure (and every 0.1% value there between). In another embodiment, an oligodendrocyte or an oligodendrocyte population is at least 50% pure, and more preferably, at least 60% pure, and more preferably, at least 70% pure, and more preferably, at least 80% pure, and more preferably, at least 90% pure. As will be understood by the skilled artisan, it is possible to specifically monitor the functions and properties of an oligodendrocyte, even when the oligodendrocyte is not within culture conditions of purity or “essentially pure.” As will be understood based on the disclosure set forth herein, when the majority of cells in a preparation are oligodendrocytes, it is possible to identify genes either up- or down-regulated during myelination, using the methods set forth herein, because there will exist an overlap in such genes identified. However, certain genes may be masked if the predominant contaminating cell type is a cell positive for adenosine receptors, such as an astrocyte.

The methods embodied herein further comprise a method of identifying a gene that is regulated during myelination of an oligodendrocyte. In an aspect, the materials and methods set forth herein include identifying a molecule, the expression of which molecule is altered in response to treatment of an oligodendrocyte with an adenosine receptor agonist. This is because it has been shown herein that an adenosine receptor agonist can be used to induce myelination of an oligodendrocyte. Such induction may involve the up- or down-regulation of at least one gene in an oligodendrocyte, and the materials and methods set forth herein provide methods and compositions useful for identification of such genes.

The materials and methods set forth herein should not be limited to the use of an adenosine receptor agonist to induce myelination, however. Rather, any substance or process that can effect myelination of an oligodendrocyte can be used, the substance or process either now known or yet to be discovered. This is because it has presently been shown, in part, that comparative methods of differentially inducing myelination can be used to identify genes that are regulated during the myelination process in an oligodendrocyte.

In one embodiment, an adenosine receptor agonist is adenosine. In another embodiment, an adenosine receptor agonist is an adenosine analog. Such analogs include, but are not limited to, MECA 9-[5(methylcarbamoyl)-β-D-ribofuranosyl]adenosine, CPA (N⁶-cyclopentadyladenosine), CHA (N⁶-cyclohexyladenosine), CCPA (2-chloro-CPA) specific for ADORA1, and pan-ADORA agonists such as NECA (N-ethylcarboxamidoadenosine and XAC (8-[4-[[[[(2-aminoethyl)amino]carbonyl]methyl]oxy]phenyl]-1,3-dipropylxanthine.

In another embodiment, an adenosine receptor agonist is an adenosine A1 receptor (“ADORA1”) agonist. It is known that ADORA1 and ADORA3 down-regulate cAMP, whereas ADORA2A and ADORA2B up-regulate it. Therefore, according to the methods set forth herein, any pan-ADORA agonist is useful with the methods and compositions set forth herein, provided that the agonist affects ADORA 1. Therefore, in another aspect, an adenosine receptor agonist is pan-active, and is able to bind to all types of adenosine receptors. In yet another aspect, an adenosine receptor agonist is able to bind to more than one type of adenosine receptor. In another aspect, an adenosine receptor agonist is specific for only one type of adenosine receptor. The skilled artisan, when armed with the disclosure set forth herein, will understand how to identify an adenosine receptor agonist useful in a method or material as described or embodied herein.

By way of a non-limiting example, an agonist specific for an adenosine A1 receptor is useful in the materials and the methods embodied herein. This is because it has been shown that the adenosine A1 receptor can be preferentially expressed under oligodendrocyte culture conditions, as described in detail elsewhere herein. In another embodiment, an agonist specific for more than one type of adenosine receptor can be used in a method embodied herein. This is again because it has been shown that the adenosine A1 receptor can be preferentially expressed under oligodendrocyte culture conditions, and therefore, such an agonist will primarily, or solely, mediate its activity through the adenosine A1 receptor.

A method embodied herein also includes a verification that an oligodendrocyte is indeed myelinating, when myelination of an oligodendrocyte is desired. In this aspect, direct detection of myelination can be used. However, it will also be understood that any method useful for verifying oligodendrocyte myelination can be used in the methods described herein. Such methods include, but are not limited to identification of even length and compact appearance of myelin using the anti-myelin basic protein (“MBP”) staining, as described in detail elsewhere herein.

In another aspect, a “surrogate marker” can be used to characterize oligodendrocyte myelination, also as described in detail elsewhere herein. In one embodiment, myelination of an oligodendrocyte is detected using a surrogate marker. In one aspect, a surrogate marker is myelin oligodendrocyte glycoprotein (MOG). Detection of MOG may be conducted using any protein detection method known in the art, including, but not limited to, detection through antibody binding to MOG. See, for example, in Harlow et al., ANTIBODIES: A LABORATORY MANUAL, COLD SPRING HARBOR LABORATORY, Cold Spring Harbor, N.Y. (1988).

As set forth in detail herein, various methods and/or compositions can be used to ascertain the up-regulation or down-regulation of the expression of a gene in an oligodendrocyte. Various methods and/or compositions can also be used to ascertain the up-regulation or down-regulation of the expression of a polypeptide from a particular gene in an oligodendrocyte.

Such information can be obtained by evaluating the level of a polypeptide in an oligodendrocyte. It should be understood that a polypeptide is considered to be “in an oligodendrocyte” whether the polypeptide is contained within the interior of the oligodendrocyte, or whether the polypeptide is integrated into the membrane of the oligodendrocyte, or displayed on the outer surface of the oligodendrocyte. However, it should also be understood that a polypeptide can be detected, quantified, and/or assayed for outside of the oligodendrocyte, such as in the cell culture medium.

Measurement of a polypeptide level or amount can be conducted using any technique now known in the art or yet to be discovered. Such techniques include, but are not limited to, antibody-based detection, amino acid sequencing, and affinity chromatography. Once armed with the knowledge of a polypeptide, the level of which is either up- or down-regulated in connection with an oligodendrocyte competent of myelination, it is well-within the skill of the ordinary artisan to determine the identity and/or sequence of the polypeptide. Based on techniques set forth in, e.g., Sambrook et al., MOLECULAR CLONING: A LABORATORY MANUAL, volumes 1-3 (3^{rd} ed., Cold Spring Harbor Press, NY 2001) or any of the prior editions by Sambrook, the sequence of the identified polypeptide can be used to develop a set of degenerate primers for panning a cDNA library representative of the genetic makeup of an oligodendrocyte, thereby identifying the gene that is regulated during myelination of the oligodendrocyte, i.e., a “target” gene.

A target gene can also be identified through nucleic acid detection or cell-biological methods. For example, RNA encoding a target gene may be identified as being up- or down-regulated during the myelination process by an oligodendrocyte. Methods include, but should not be limited to, interfering RNA (“RNAi”) techniques, or nucleic acid expression systems, such as lentiviral or adenoviral expression systems. One advantage of a viral expression system is the potential to selectively and permanently inhibit expression of a gene. In one aspect, an interfering RNA (“RNAi”) technique is used to down-regulate a gene. Another advantage of a viral expression system is the potential to specifically over-express a gene, such as is useful to validate an up-regulated target.

In one embodiment, a target gene is identified using a nucleic acid detection method. In one aspect, total RNA is isolated from an oligodendrocyte, as described in detail elsewhere herein, and the RNA isolated there from subsequently characterized and quantified. Differential analysis of RNA between one oligodendrocyte population and another provides the basis for the comparison of up- or down-regulated target genes in this embodiment. Such techniques are described in detail elsewhere herein.

2.2 Methods of Myelinating and Modulating Myelination of Oligodendrocytes

The materials and methods set forth herein provide methods and compositions for modulation of the myelination of an oligodendrocyte. This is because, as demonstrated for the first time herein, target genes can now be specifically identified, wherein the up- or down-regulation of such genes in an oligodendrocyte is correlated with the competence of the oligodendrocyte to myelinate.

In one embodiment, inhibition of a target gene in an oligodendrocyte is used to stimulate the oligodendrocyte to become a myelinating oligodendrocyte. In another embodiment, inhibition of a target gene in an oligodendrocyte is used to inhibit myelination by the oligodendrocyte. In yet another embodiment, stimulation of a target gene in an oligodendrocyte is used to stimulate the oligodendrocyte to become a myelinating oligodendrocyte. In still another embodiment, stimulation of a target gene in an oligodendrocyte is used to inhibit myelination of the oligodendrocyte.

In an embodiment, stimulation of myelination of an oligodendrocyte by way of either stimulation or inhibition of a target gene is used to induce the capacity to myelinate in oligodendrocytes. In one aspect, such stimulation or inhibition of a target gene is used to re-myelinate the axon of one or more neurons. In another aspect, the myelin sheath of at least one axon of a neuron is re-established after a de-myelinating event, using the disclosed materials and methods. In one embodiment, a demyelinating event is MS. In another embodiment, a demyelinating event occurs in connection with a myelinating disease or disorder, as described in detail elsewhere herein.

In yet another embodiment, stimulation of myelination of an oligodendrocyte by way of stimulation or inhibition of a target gene in an oligodendrocyte is used to newly myelinate a neuron. Unstimulated oligodendrocytes fail to myelinate axons. The stimulation of the “early” oligodendrocyte leads to a different differentiation path that enables the oligodendrocyte to myelinate axons. An oligodendrocyte in a lesion is typically a new oligodendrocyte, and not an old oligodendrocyte that now can be induced to myelinate again. That is, typically, demyelination leads to oligodendrocytic cell death). In one aspect, such stimulation or inhibition is used to provide new myelination of the axon of one or more neurons.

The present materials and methods also provide for modulation of sphingosine kinase. This is because, as demonstrated for the first time herein, sphingosine kinase is down-regulated in myelinating oligodendrocytes. Sphingosine kinase is a key regulatory enzyme in a variety of cellular responses. Its activity can affect inflammation, apoptosis and cell proliferation. Sphingosine kinase is therefore an important target for therapeutic intervention. In one embodiment, inhibition of sphingosine kinase in an oligodendrocyte is used to stimulate myelination of the oligodendrocyte. In one aspect, the inhibition of sphingosine kinase is conducted in the absence of adenosine A1 receptor stimulation. In another aspect, the inhibition of sphingosine kinase is conducted in conjunction with adenosine A1 receptor stimulation.

In another embodiment, stimulation of myelination of an oligodendrocyte by way of sphingosine kinase inhibition is used to remyelinate an oligodendrocyte. In one aspect, such inhibition of sphingosine kinase is used to re-myelinate the axon of one or more neurons. In another aspect, the myelin sheath of at least one axon of a neuron is re-established after a de-myelinating event. In one embodiment, a demyelinating event is MS. In another embodiment, a demyelinating event occurs in connection with a myelinating disease or disorder, as described in detail elsewhere herein.

In yet another embodiment, stimulation of myelination of an oligodendrocyte by way of sphingosine kinase inhibition is used to newly myelinate an oligodendrocyte. In one aspect, such inhibition of sphingosine kinase is used to provide new myelination of the axon of one or more neurons.

The present materials and methods also provide for treating a demyelinating disease in a mammal comprising administering to the mammal a sphingosine kinase modulator. In an embodiment, the demyelinating disease is MS. In another embodiment, the mammal is a human. In one aspect, the sphingosine kinase modulator is an antagonist of sphingosine kinase. In one embodiment, a sphingosine kinase antagonist is used to down-regulate sphingosine kinase expression and/or activity, thereby enhancing myelination by an oligodendrocyte. In another aspect, the sphingosine kinase modulator is an agonist of sphingosine kinase.

In another embodiment, a method for treating a demyelinating disease in a mammal by way of sphingosine kinase modulation is used to remyelinate an oligodendrocyte. In one aspect, such modulation of sphingosine kinase is used to re-myelinate the axon of one or more neurons. In another aspect, the myelin sheath of at least one axon of a neuron is re-established after a de-myelinating event. In one embodiment, a demyelinating event is MS. In another embodiment, a demyelinating event occurs in connection with a myelinating disease or disorder, as described in detail elsewhere herein.

The present materials and methods also provide for myelinating an oligodendrocyte in a mammal comprising administering to the mammal a sphingosine kinase modulator. In one aspect, the sphingosine kinase modulator is an agonist of sphingosine kinase. In another aspect, the sphingosine kinase modulator is an antagonist of sphingosine kinase.

It will be understood, based on the disclosure set forth herein, that any compound that can act as an agonist or antagonist of sphingosine kinase is useful in the materials and methods embodied herein. Such compounds include, but are not limited to, siRNA, dsRNA, antisense DNA, as well as compounds such as NN-dimethylsphingosine (DMS). See, e.g., Cuvillier, 2002 Biochim. Biophys Acta. 1585:153-162 and Baumruker et al., 2005 Immunol. Lett. 96:175-185.

Furthermore, it will be understood, based on the disclosure set forth herein, that any composition or method useful in down-regulating sphingosine kinase expression or activity in an oligodendrocyte is useful in the materials and methods embodied herein. Such methods and/or compositions include, but should not be limited to, RNAi, viral vector systems, and cell-permeable antagonists, among others.

2.3 Cell Culture Systems for Modeling MS and Other Myelinating Diseases

Also provided is a cell culture system for modeling MS. This is because it has been demonstrated herein that a cell culture system according to the methods and compositions embodied herein can be used to identify a gene that is regulated during myelination of an oligodendrocyte. Characterization of such genes which are up- or down-regulated during MS pathology can provide a target for therapeutic intervention, to prevent loss of neuronal myelin and/or to generate remyelination of neurons that have lost myelin. Similarly, a cell culture system is provided for modeling other myelinating diseases, wherein such diseases are related to the up- or down-regulation of target genes involved with oligodendrocyte myelination.

In an embodiment, a cell culture system is provided which is useful for identifying a gene that is regulated during myelination of an axon by an oligodendrocyte. In an embodiment, the culture system includes at least two oligodendrocytes, wherein the oligodendrocytes are differentiated in vitro to the state of myelin protein expression. The first oligodendrocyte is differentiated in the presence of an adenosine receptor agonist and the second oligodendrocyte is differentiated in the absence of said adenosine receptor agonist, for the purpose of comparing the gene expression profiles of the oligodendrocytes. The cell culture system enables the identification of a molecule, the expression of which molecule is altered in response to treatment of an oligodendrocyte with an adenosine receptor agonist. The altered expression of such a molecule is an indication that the oligodendrocyte in which the expression is altered is competent to form compact myelin. In this way, a culture system as set forth herein enables the identification of a gene or gene product, wherein the gene is regulated during myelination of an oligodendrocyte.

In an aspect of the materials and methods set forth herein, a cell culture system is useful for drug screening. This is because a cell culture system as set forth herein can be used to identify the effect of a molecule on an oligodendrocyte, and in particular, on the oligodendrocyte competent for myelination. In an embodiment, the culture system includes at least two oligodendrocytes, wherein the oligodendrocytes are differentiated in vitro to the state of myelin protein expression. The first oligodendrocyte is differentiated in the presence of a pre-selected molecule and the second oligodendrocyte is differentiated in the absence of the pre-selected molecule, for the purpose of comparing the phenotypic and/or genotypic profiles of the oligodendrocytes. The cell culture system thereby enables the identification of a molecule which has the ability to modulate the capacity of an oligodendrocyte to myelinate, by way of a differential comparison of a treated and a non-treated oligodendrocyte.

Methods and compositions useful in a culture system as set forth herein are set forth in detail elsewhere herein, and will therefore not be described extensively at this point. Similarly, methods of optimizing such culture systems will be understood by the skilled artisan in view of the disclosure set forth herein, including the experimental examples, and will therefore not be addressed in detail.

A culture system as set forth herein will be understood to be any culture system useful for maintaining, growing, proliferating, or differentiating oligodendrocytes. Such conditions include, but are not limited to, those set forth in detail in the experimental examples described herein.

### EXAMPLES

Although the present invention has been described in detail with reference to examples below, it is understood that various modifications can be made without departing from the spirit of the invention, and would be readily known to the skilled artisan.

### Example 1

### Development of Cell Culture Systems to Study Myelination

In order to address the myelinating and demyelinating aspects of the secondary progressive stage of MS, a cellular system was established to represent such MS lesions. A co-culture system of purified rat retinal ganglion cells (RGC) and optic nerve oligodendrocytes was used. This system recapitulates some significant aspects of MS lesions, namely, the oligodendrocytes loosely wrap membrane around axons, but fail to form internodes of compact myelin. The RGC neurons in these cell cultures are, for the most part, electrically inactive and may resemble dystrophic neurons observed in MS lesions. Furthermore, cell isolation, purification, and culture methods, conditions and compositions were developed, as described herein.

Purification of optic nerve oligodendrocytes was conducted as follows. Optic nerve oligodendrocytes are purified from postnatal day 5 (p5) Sprague-Dawley (“SD”) rat pups according to the methods described in Meyer-Franke et al. (1999 Mol. Cell Neurosci 14:385-397).

Optic nerves were dissected and cut into small pieces. Nerves were then digested in a papain/DNAse solution in phosphate-buffered saline (PBS), containing sodium pyruvate and glucose (Invitrogen), 33 units of papain/ml and 1,000 units DNAse/ml PBS (both enzymes from Worthington) for 1 hour and 45 minutes at 37° C. After the digest, optic nerve pieces were briefly washed and a solution containing 1.5 mg/ml Trypsin inhibitor from hen egg white (Roche Biochemicals) and 1.5 mg/ml BSA (Sigma) was added. Pieces were triturated twice in 1 ml with the above solution through a 25-guage needle, centrifuged at low speed between triturations, and then triturated through a 23-guage needle several times until the pieces fell apart.

Subsequently the cell suspension was washed with a PBS solution containing 15 mg/ml Trypsin inhibitor and 15 mg/ml BSA. Cells were then transferred into ‘panning buffer’ containing PBS+0.02% BSA and 5 μg/ml insulin (Sigma) and incubated on the panning plates.

Panning plates were prepared as follows. Secondary antibodies used included affinity-purified goat anti-mouse IgM (mu chain-specific) and affinity-purified goat anti-mouse IgG (H+L chain-specific). Primary monoclonal antibodies used included the A2B5 antibody (IgM; ATCC) and the anti-RAN-2 antibody (IgG; ATCC). Petri dishes (100×15 mm; Falcon) were incubated with 10 ml of Tris buffer solution (50 mM; pH 9.5) containing 10 μg/ml secondary antibody, either anti-IgM or anti-IgG, for 12 hours at 4° C. Each dish was washed three times with 8 ml of PBS and incubated with 12 ml A2B5 hybridoma supernatant (one IgM dish) and 12 ml of anti-RAN-2 supernatant for at least 1 hour at room temperature. The antibody solution was removed, the plates washed were three times with PBS, and PBS was left on the plates until subsequent use.

The immunopanning procedure used was as follows. The optic nerve cell suspension was resuspended in 7 ml of PBS containing insulin (5 μg/ml; Sigma) and filtered through a 40 micron filter insert (Falcon/Becton-Dickinson). To deplete type 1 astrocytes and meningeal cells (as well as microglia and macrophages, which adhere through Fc receptors to the first immunoglobulin-coated panning dish used), the cell suspension was first placed on the RAN-2 plate for 30 minutes at room temperature, with brief and gentle agitation after 15 minutes. The nonadherent cells were transferred to the second RAN-2 plate for a 30 minute incubation, after which the nonadherent cells were transferred to the A2B5 dish to select for the A2B5+oligodendrocyte progenitor cells. After 45 minutes, this plate was washed five times with 5 ml of PBS in order to remove the nonadherent cells and adherent cells were taken off the plates after 10 minutes incubation at 37° C. with 535 units of Trypsin/ml (Worthington) in Earl's Balanced Salt Solution (EBSS; Invitrogen). After a wash in 30% FBS in PBS to block Trypsin, cells were counted and plated at the appropriate density.

The culture conditions for the immunopanning procedure was as follows. The A2B5 positive progenitor cells were cultured on a polyethyleneimine substrate (PEI; Sigma) in defined medium [DMEM (Invitrogen) containing Sato supplement, insulin (5 μg/ml; Sigma), PDGF (20 ng/ml) and NT3 (20 ng/ml) (Preprotech)] that encouraged mitogenesis for 72 hours. Sato supplement contained transferrin (Sigma) at 5.6 ng/ml, BSA (crystalline; Sigma) at 100 μg/ml, progesterone (Sigma) at 60 ng/ml, putrescine (Sigma) at 16 μg/ml, and sodium selenite (Sigma) at 40 ng/ml. Subsequently, cells were switched to defined differentiation medium/growth medium (DMEM containing Sato supplement, B27 supplement (Invitrogen), CNTF (20 ng/ml) and NT3 20 ng/ml) (Preprotech)).

Cortical oligodendrocytes were purified as follows. Cortices were dissected from postnatal day 1-2 old SD rat pups, cut into small pieces and digested in a papain/Dnase solution in PBS (16.5 units papain/ml PBS and 5,000 units Dnase/ml PBS; Worthington Biochemical Corporation) for 45 minutes at 37° C. The cortices were then gently triturated in a PBS solution with a pipette, the solution containing 1.5 mg/ml trypsin inhibitor from hen egg white (Roche Biochemicals) and 1.5 mg/ml BSA (Sigma). The cell suspension was briefly washed in PBS solution containing 15 mg/ml trypsin inhibitor and 15 mg/ml BSA. The cells were then incubated on two subsequent RAN-2-panning dishes and a final A2B5 dish (4 corteces per one set of plates). The cells were then plated on PEI coated dishes at low density in defined medium containing insulin (5 μg/ml; Sigma), PDGF, and NT3. Under those conditions, the majority of cells that adhered to the plates were the A2B5+progenitor cells which will divide for 72 hours. The medium was then switched to differentiation medium (as described above). This method provided cultures with about 80% oligodendrocytes, and about 20% contaminating cells (primarily astrocytes and a few neurons).

Culture conditions used for the cortical oligodendrocytes were as follows. The A2B5 positive progenitor cells were cultured on a polyethyleneimine (PEI) substrate in defined medium [DMEM (Invitrogen) containing Sato supplement, PDGF (20 ng/ml), and NT3 (20 ng/ml; Preprotech), which medium encouraged mitogenesis, for 72 hours. Subsequently, the cells were switched to a defined differentiation medium/growth medium (DMEM containing Sato supplement, B27 supplement (Invitrogen), CNTF (20 ng/ml) and NT3 20 ng/ml) (Preprotech)).

Retinal ganglion cells (RGCs) were purified and cultured according to Meyer-Franke et al. (1995 Neuron 15:805-819). Retinae were dissected from postnatal day 5 (p5) SD rat pups. Cells were digested in a papain/Dnase solution in phosphate-buffered saline (PBS), containing sodium pyruvate and glucose (Invitrogen), including 16.5 units papain/ml and 1,000 units Dnase/ml PBS (both enzymes from Worthington), for 30 minutes at 37° C. After the digestion, retinae are briefly washed and a solution containing 1.5 mg/ml Trypsin inhibitor from hen egg white (Roche Biochemicals) and 1.5 mg/ml BSA (Sigma), including anti-rat-macrophage antiserum (Axell/Accurate; 1:100). The retinae were gently triturated and the cell suspension incubated for 15 minutes prior to centrifugation at 200×g for 15 minutes. Cells were then washed with a PBS solution containing 15 mg/ml Trypsin inhibitor and 15 mg/ml BSA. Cells were spun at 200×g for 15 minutes and the cell pellet then resuspended in ‘panning buffer’ containing PBS+0.02% BSA and 5 μg/ml insulin (Sigma). The cell suspension was then incubated on panning plates, described as follows.

Panning plates were prepared as follows. Secondary antibodies used included affinity-purified goat anti-mouse IgM (mu chain-specific; Jackson) and affinity-purified goat anti-rabbit IgG (H+L chain-specific; Jackson). Primary antibodies used included monoclonal supernatant IgM antibody against mouse Thy1.1 (T11D7e2, American Type Culture Collection, ATCC, TIB 103). Petri dishes (two 150×15 mm and one 100×15 mm; Fisher) were treated with 5-15 ml of Tris buffer solution (pH 9.5) with 10 pg/ml secondary antibody (anti-mouse IgM on the small dish or anti-rabbit IgG on the large dishes) for 12 hours at 4° C. The dishes were washed three times with 8 ml of phosphate buffered saline (PBS), and the small dish was further incubated with 8 ml of Thy 1.1 IgM monoclonal supernatant for 2 hours at room temperature. The supernatant was removed, and the plate was washed three times with PBS. To prevent nonspecific binding of cells to the panning dish, 5-15 ml of PBS with 2 mg/ml BSA (Sigma) was placed on the plates for 20 minutes.

The panning procedure was conducted as follows. The retinal cell suspension was incubated on a 150 mm anti-rabbit IgG panning plate at room temperature for 45 minutes. The plate was gently swirled every 15 minutes to ensure access of all cells to the surface of the plate. If cells from more than eight retinae were panned, the non-adherent cells were transferred to another 150 mm anti-rabbit IgG panning plate for another 30 minutes. The non-adherent cells were removed with the suspension, filtered through a 70 micron filter (Falcon/Becton-Dickinson), and placed on the TllD7 panning plate. The cells were incubated on the plate as described above. After 45 minutes, plates were washed eight times with 10 ml of PBS and swirled moderately vigorously to dislodge non-adherent cells. Progress of non-adherent cell removal was monitored under the microscope. The washing was terminated when only adherent cells remained.

Adherent cells were removed from the plate as follows. Four milliliters of a trypsin solution (535 units/ml) was prepared by diluting a trypsin 20× stock (Worthington) into EBSS. Cells on the panning dish were incubated with this solution for 10 minutes in a 10% CO₂ incubator at 37° C. The cells were dislodged by gently pipetting trypsin solution around the plate. Ten milliliters of a 30% fetal calf serum solution were added to inactivate the trypsin, and the cells were centrifuged and collected as described above.

The culture conditions for RGC culture were as follows. RGCs were cultured on a PDL/merosin (2 μg/ml; Chemicon) substrate in defined medium (Neurobasal™; Invitrogen) containing Sato supplement, B27™ supplement, insulin (5 μg/ml), brain-derived neurotrophic factor BDNF (50 ng/ml) and CNTF (20 ng/ml) (Preprotech), with 1-10 μM forskolin (Sigma).

The astrocyte-conditioned medium used was as follows. Astrocytes were prepared from cortices of postnatal day 1-old SD rat pups. Cortices were dissected, cut into small pieces and digested in a papain/Dnase solution in PBS (16.5 units papain/ml PBS and 5,000 units Dnase/ml PBS; Worthington Biochemical Corporation) for 45 minutes at 37° C. The cortices were then gently triturated in a PBS solution containing 1.5 mg/ml trypsin inhibitor from hen egg white (Roche Biochemicals) and 1.5 mg/ml BSA (Sigma). The cell suspension was briefly washed in PBS solution containing 15 mg/ml trypsin inhibitor and 15 mg/ml BSA. The cell pellet was resuspended in DMEM containing 10% FBS and plated into PDL coated T75 flasks (Falcon/Becton-Dickinson). After 7 days in culture, oligodendrocytes were shaken loose and the cell monolayer containing the astrocytes and neurons was trypsinized with a 0.25% Trypsin/EDTA solution (Invitrogen) and replated into fresh PDL-coated T75 flasks at a distribution of 2 flasks for every one primary flask. The cells that proliferated were astrocytes, and were kept in culture for prolonged periods of time, as necessary. For the myelination cultures, the culture medium was switched to DMEM with 0.5% FBS.

Co-cultures of RGCs and optic nerve oligodendrocytes were prepared and used as follows. Purified oligodendrocytes were plated on PDL/merosin (Chemicon) substrate-coated tissue culture dishes in defined DMEM Sato medium, promoting proliferation for two days. After 2 days, the medium was switched to defined DMEM Sato medium, which contained factors to promote differentiation. Twenty-four hours after the switch to differentiation medium, ADORA1 agonists were added. Retinal ganglion cells were plated 24 hours thereafter, and the medium consisted of 50% astrocyte-conditioned medium (method see above) with DMEM Sato medium mixed with fresh defined Neurobasal™ (Invitrogen) Sato medium, containing B27™ supplement and growth factors (BDNF and CNTF), to promote retinal ganglion cell survival and 0.5% FBS. The FBS was added to help retinal ganglion cell survival. This is because forskolin, which up-regulates cAMP, could not be used when adding any ADORA agonists or antagonists, since these receptors are G-protein-coupled receptors and therefore, regulators of cAMP themselves. Cultures were grown for two weeks, with medium changes occurring every three days.

In control conditions, oligodendrocytes loosely wrap membrane around axons, often around a whole axon bundle, but they would not form internodes of myelin. However, in the presence of adenosine, approximately 90% of oligodendrocytes established what appeared to be segments of myelin on individual axons within an axon bundle. These results demonstrated that a signal like adenosine, which is normally secreted from electrically active neurons, can lead to robust myelination in this culture system. Adenosine appears to trigger a crucial switch in oligodendrocytes that enable them to myelinate.

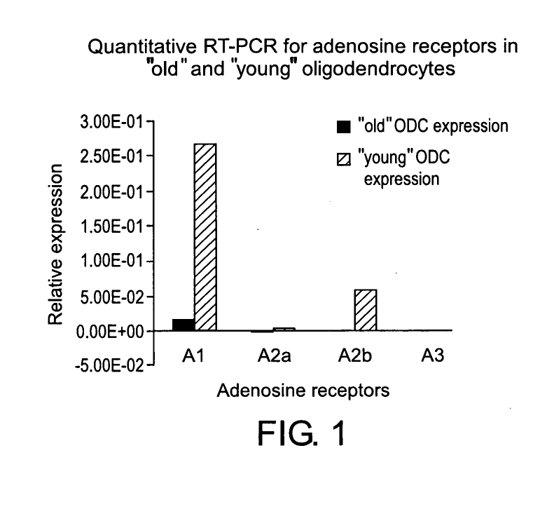

Electrically active neurons secrete factors such as adenosine to stimulate oligodendrocytes to myelinate (Stevens et al., 2002 Neuron 36:855-868). In mouse cortical oligodendrocytes, all four adenosine receptors (i.e., A1, A2a, A2b, A3) have been described. Adenosine receptors are G-protein coupled receptors (GPCRs) with opposite effects on cellular cAMP levels. While stimulation of A1 and A3 will lower cAMP, activation of A2a and A2b will increase cAMP levels. Quantitative RT-PCR was used to characterize which adenosine receptor is mediating the switch committing rat optic nerve oligodendrocytes to myelinate. Expression of adenosine receptors had been demonstrated previously and protein expression had been analyzed by Western blot analysis. However, as illustrated in FIG. 1, only ADORA1 and a low level of ADORA2B were found to be present on the optic nerve oligodendrocytes. The following primers were used for the experiments illustrated in FIG. 1:

| | |
|---|---|
| A1: | |
| 5′ primer: AACATTGGGCCACAGACCTACT | (SEQ ID NO:1) |
| 3′ primer: GCAGCACCCAGACGAAGAAG | (SEQ ID NO:2) |
| A2a: | |
| 5′ primer: CGCCATCGACCGCTACATC | (SEQ ID NO:3) |
| 3′ primer: GGGCCGCCAGAAAAATCC | (SEQ ID NO:4) |
| A2b: | |
| 5′ primer: GACGTGGCTGTGGGACTCTTC | (SEQ ID NO:5) |
| 3′ primer: CACAGGGCAGCAGCTCTTATTC | (SEQ ID NO:6) |
| A3: | |
| 5′ primer: AAGCCAACAATACCACGACGAG | (SEQ ID NO:7) |
| 3′ primer: AGCAGAGGCCCAGGAATAGC. | (SEQ ID NO:8) |

Expression of mRNA was analyzed by quantitative RT-PCR to obtain a quantitative measure of expression and to enable investigation of the regulation of receptor subtypes during oligodendrocyte differentiation. Oligodendrocytes were isolated from rat optic nerves and grown in mitogenic media for 3 days, mitogenic media was then replaced with differentiation media and cells harvested for RNA isolation on day 1 (early stage) and day 5 (late stage) post-differentiation. Total RNA isolation was conducted using Ambion's total RNA isolation kit as per protocol. Quantitative RT-PCR was set up as a 2-step reaction following the protocol in the Applied Biosystems protocol book. Primers and probes for PCR were designed using Primer Express software, TaqMan™ probe with 6FAM as the fluorophore at the 5′ end and TAMRA as quencher at the 3′ end.

Primers and probes were designed using the Primer Express software from Applied Biosystems. Probes were designed with 6-FAM as fluorophore at the 5′end and TAMRA as quencher at the 3′ end. QRT-PCR reactions were set up as a one-step reaction using 20 ng of total RNA as template, and included 1× Taqman™ buffer, 5 mM MgCl₂, 1.5 mM dNTP mix, 300 nM forward and reverse primers, 150 mM Taqman™ probe, 20 units RNase inhibitor, 5 units Multiscribe™ MuLV reverse transcriptase and 2 units AmpliTaq™ Gold DNA polymerase in a 50 microliter reaction. All reagents used were from Applied Biosystems. Reactions were run on a ABI 7500 using the following cycling conditions: “Hold” at 48° C. for 30 minutes, “Hold” at 95 for 10 minutes, “Denature” at 95° C. for 15 seconds, and “Anneal/Extend” at 60° C. for 1 minute. The last two steps were repeated for 40 cycles.

As illustrated in FIG. 1, A1 is the dominant adenosine receptor form expressed in oligodendrocytes, in both the early and late stages of oligodendrocyte differentiation. A1 expression was observed to be down-regulated during the late stage of differentiation. This corresponds to results set forth elsewhere herein, whereby in vitro myelination cultures of oligodendrocytes are more responsive to adenosine at the early stage of differentiation with a significant drop in adenosine responsiveness at late stages of differentiation as measured by percentage of myelination.

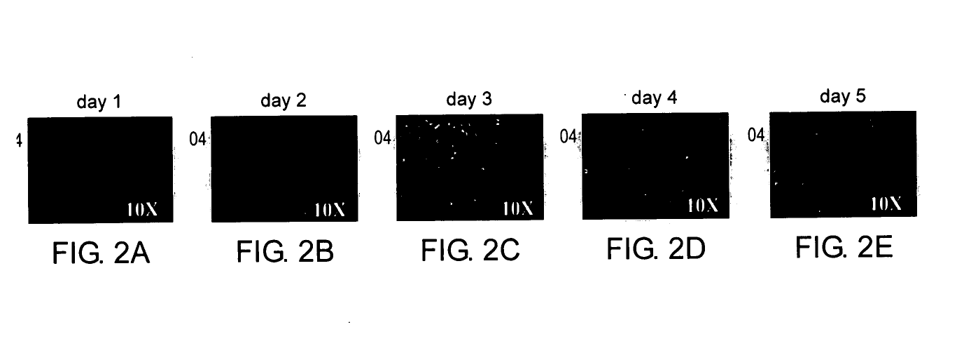

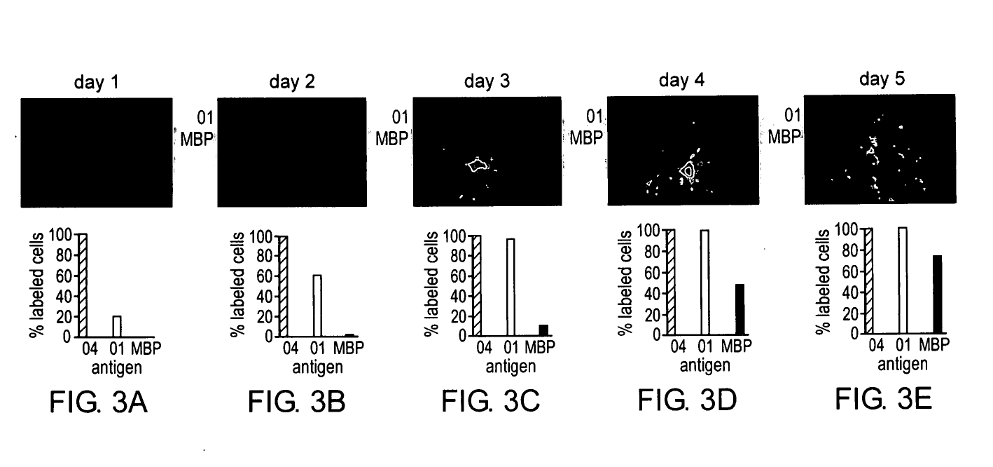

Further, rat optic nerve oligodendrocytes were analyzed for the expression of differentiation markers for 5 days after the oligodendrocytes were switched to differentiation medium. The markers that were used in order for their expression during differentiation were O4 (sulfatide), O1 (galactocerebroside), and Myelin Basic Protein (MBP). As shown in FIG. 2, all cells were positive for 04 on day 1 after the switch to the differentiation medium. The majority of cells still had the bipolar morphology typical for very young oligodendrocytes. Through day 5, these oligodendrocytes changed dramatically in size and shape. Cell processes changed from thin spiny processes around the cell body on day 2 to almost leaf-like processes on day 5. As shown in FIG. 3, on day 2, approximately 60% of the O4 positive oligodendrocytes expressed the O1 antigen, and by day 3, all oligodendrocytes were O4 and O1 positive. About 10% of these were positive for MBP, as well. On day 4, about 45% of the oligodendrocytes were expressing MBP, and by day 5, this number increased to 80%. Cell shape was observed to show the same morphology, regardless of whether the cells were stimulated to a myelinating phenotype or not. This is because there is a default pathway leading to expression of myelin proteins, which has nothing to do with capability to myelinate or not of an oligodendrocyte.

### Example 2

### Use of siRNA as a Control for Oligodendrocyte Cell Culture Screening and Modulation of Myelination

It was also demonstrated herein that oligodendrocytes are competent for RNAi techniques. The criterion for choosing an endogenous target for RNAi requires that the target is relatively non-essential for oligodendrocyte survival and functioning. To accomplish this, the endogenously expressed cell adhesion molecule NCAM was selected for preliminary validation. Four RNAi oligonucleotides for NCAM were prepared (Qiagen). Oligodendrocytes were grown in mitogenic media for 3 days and differentiated for two days before transfecting with siNCAM (“siNCAMs 1-4,” which represent a set of four different siRNAs that were evaluated for use in RNAi experimentation as described herein).

The protocol for siRNA transfection into oligodendrocytes is as follows. All transfections were conducted using 24-well plates, The first solution included 50 μL OptiMEM medium, 0.8 μg plasmid DNA and/or 500 ng siRNA (10 nM or 100 nM final concentration). The second solution included 50 μL OptiMEM medium and 1.5 μL LF2K (Invitrogen). Each mixture was allowed to incubate at room temperature for five minutes. The two solutions were then combined, mixed, and incubated at room temperature for 20 minutes. Medium was then removed from the cells and replaced with 500 μL of fresh differentiation medium. The complex was then added to the medium on the cells, mixed, and incubated for 3 hours at 37 C, with 10% CO₂. The medium was changed to differentiation medium, and the cells incubated for 24 hours to 48 hours.

The transfection reagent used was Lipofectamine 2000™ (Invitrogen; Carlsbad, Calif.). Oligodendrocytes were incubated with transfection reagent for 3 hours followed by a media change and were then allowed to incubate either 24 or 48 hours post-transfection. Expression of NCAM was determined by immunostaining with anti-NCAM antibody (Chemicon) as well as by Quantitative RT-PCR (QRT-PCR) in separate experiments (see methods set forth above).

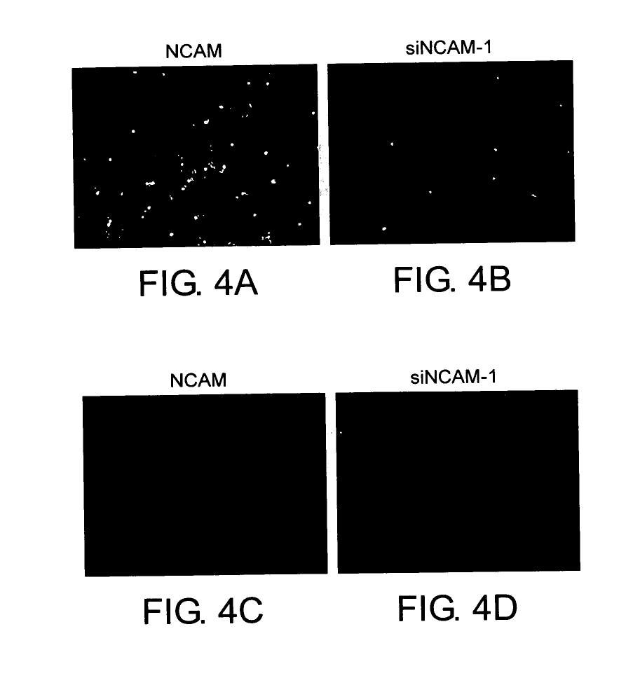

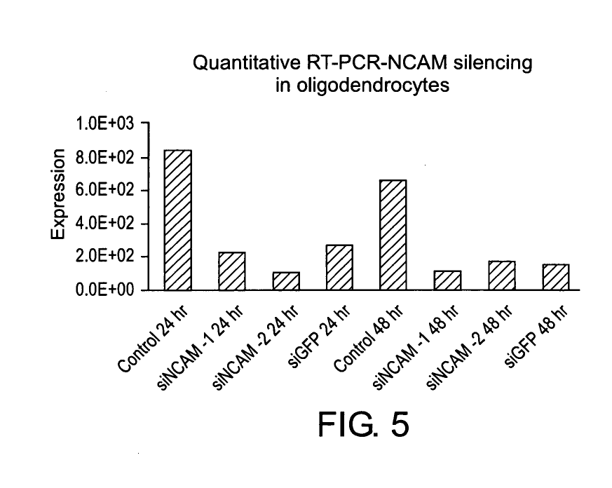

As illustrated in FIGS. 4a and 4b, siNCAM inhibits the expression of NCAM significantly compared to control 24 hours post transfection. The control experiment was conducted without siRNA, but using the same transfection medium and conditions. At 48 hours post transfection, no significant difference was observed with only a small number of cells showing a silencing effect. Another important observation from the immunostaining experiment was that almost all the oligodendrocytes appeared to take up RNAi, evidenced by the silencing effect at 24 hours, indicating feasibility of RNAi for target validation once targets have been identified from gene expression studies and for use as controls in high throughput screening of test reagents that modulate myelination using this cell culture system. In order to quantitate the silencing effect, Quantitative RT-PCR was used to study expression changes in siNCAM treated oligodendrocytes (FIG. 5). RNA was isolated at 24 hours and 48 hours post-transfection. Based on Quantitative RT-PCR results, an 80% to 90% reduction in NCAM expression was observed upon treatment of oligodendrocytes with siNCAM, confirming results with immunostaining and showing effective silencing.

### Example 3

### In Vitro Myelination Assay

Optic nerve or cortical oligodendrocytes were purified from postnatal day 2-day 5 old rat pups by immuno-panning with the A2B5 antibody (ATCC). Retinal ganglion cells were purified from 5 day old rats by immunopanning with the Thy1 antibody T11D7.

Purified oligodendrocytes were plated on PDL/merosin substrate-coated tissue culture dishes in defined DMEM Sato medium, promoting proliferation for two days. After 2 days, the medium was switched to defined DMEM Sato medium, containing factors promoting differentiation. Twenty-four hours after the switch to differentiation medium, ADORA1 agonists were added. Retinal ganglion cells were plated 24 hours thereafter, and medium consisted of astrocyte-conditioned medium with DMEM Sato medium mixed with defined Neurobasal™ Sato medium, containing growth factors promoting retinal ganglion cell survival and 0.5% FBS.

Cultures were grown for two weeks, with medium changes occurring every three days. Control co-cultures were devoid of myelin segments, whereas oligodendrocytes co-cultures with ADORA1 agonist contained myelin segments originating from approximately 90% of the oligodendrocytes in these cultures.

### Example 4

### Characterization of Nerve Myelination Cultures

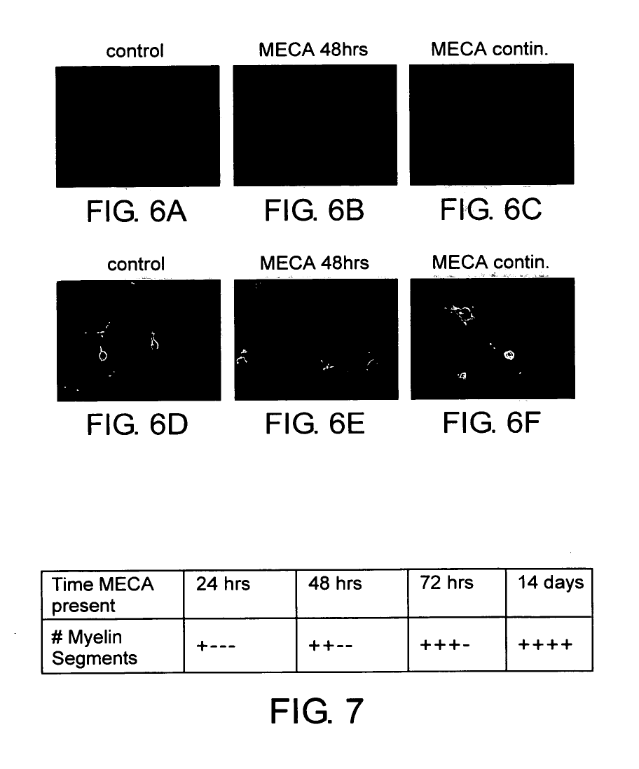

To address whether the effect of the adenosine 1 receptor (ADORA1) agonist only affects oligodendrocytes, or also affects retinal ganglion cells (RGCs) to lead to myelination, purified rat optic nerve oligodendrocytes were plated on PEI-pre-coated culture dishes (6-well plates, Falcon BD) in mitogenic medium and cultured for two days. After two days, oligodendrocytes were switched to differentiation medium and the agonist MECA (Sigma) was added at a 200 μM concentration to this culture medium 24 hours later. The agonist was removed by washing the cells at 24, 48 and 72 hours, respectively, after its administration. Oligodendrocytes were subsequently taken off the PEI plates by incubation with Trypsin (535 units/ml) in Earle's Balanced Salt Solution (EBSS; Invitrogen) at 37° C. for 5 minutes, washed with PBS containing 30% FBS (Hyclone) and were plated onto RGCs grown for one week on PDL/merosin substrate. Cultures were fixed for immunohistochemistry two weeks after co-plating the oligodendrocytes with the RGCs. Since the oligodendrocytes were pre-incubated separately with the ADORA1 agonist before they were added to the RGCs, the myelin figures observed in FIG. 6 are due to the switch in the oligodendrocytes themselves from a non-myelinating to a myelinating phenotype.

Additionally, it was determined how much time the agonist must be present in order to mediate the switch from a non-myelinating oligodendrocyte to a myelinating oligodendrocyte. As illustrated in FIG. 7, the ADORA1 agonist MECA needs to be present for at least about 72 hours to obtain a majority of myelinating oligodendrocytes. MECA was used at a concentration of 200 μM. The optimal amount of myelin figures is observed if MECA is present throughout the two weeks the oligodendrocytes are co-cultured with RGCs. It is possible that the stimulation of the A1 receptor is needed beyond the initial switch of the non-myelinating oligodendrocyte to a myelinating one.

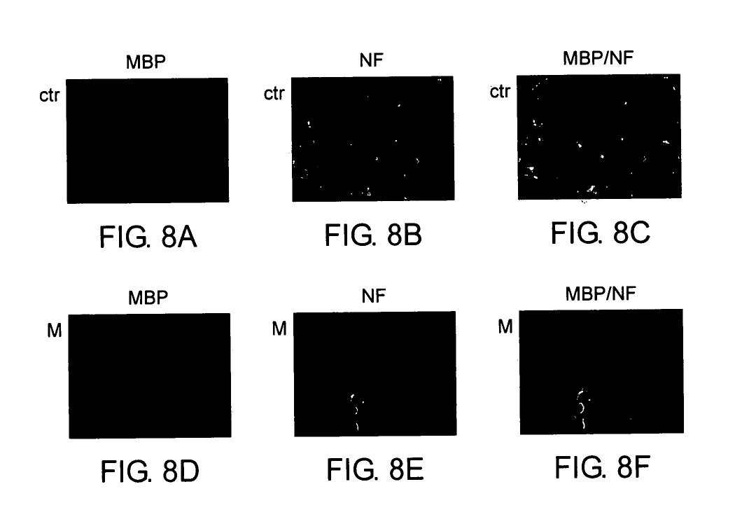

To address the question of whether it is a permanent or a reversible switch if the oligodendrocytes are cultured without neurons, oligodendrocytes were treated with ADORA1 agonist for 72 hours and kept in culture alone for another 5 days. Cultured oligodendrocytes were then removed from the PEI-coated plate and co-cultured with RGCs grown for one week in culture. Cultures were fixed as described above two weeks later. As illustrated in FIG. 8, the switch in oligodendrocytes from a non-myelinating to a myelinating phenotype is a permanent switch. MECA-stimulated oligodendrocytes would still form myelin segments even though they had not been co-cultured with neurons for a week and had not received MECA beyond the first 72 hours.

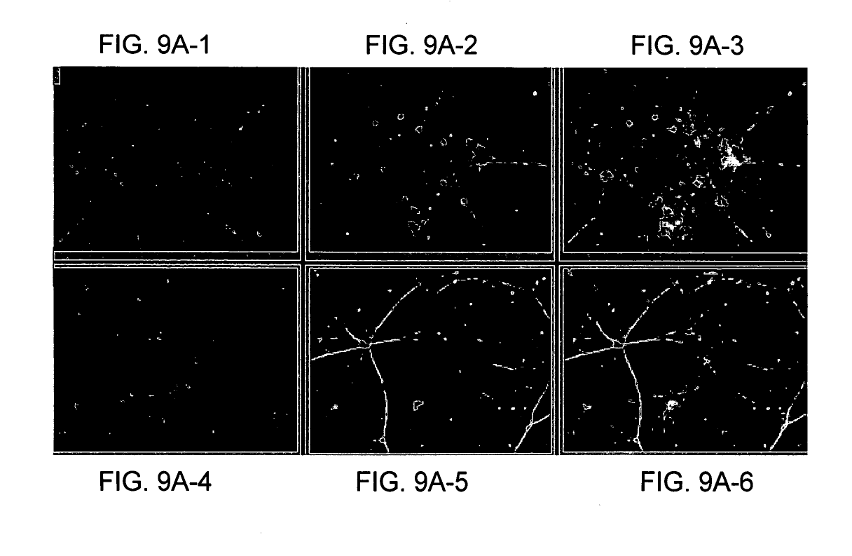

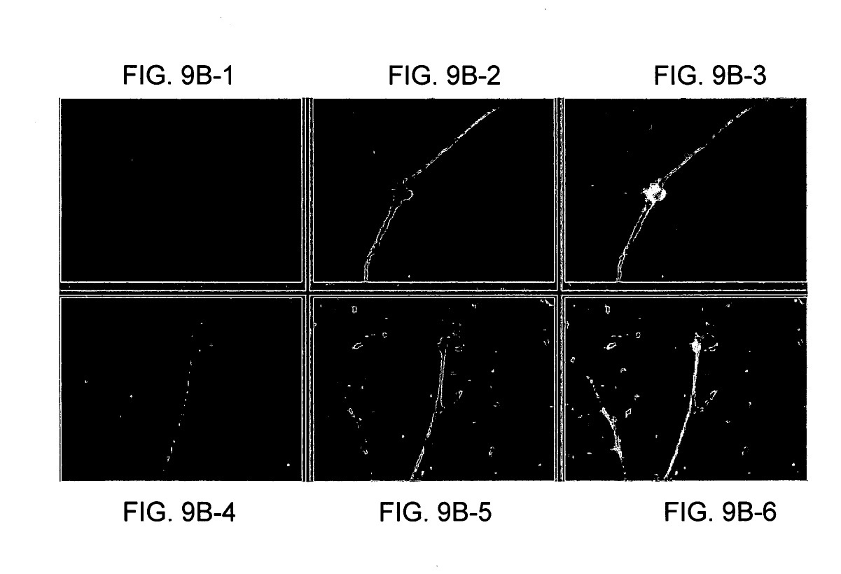

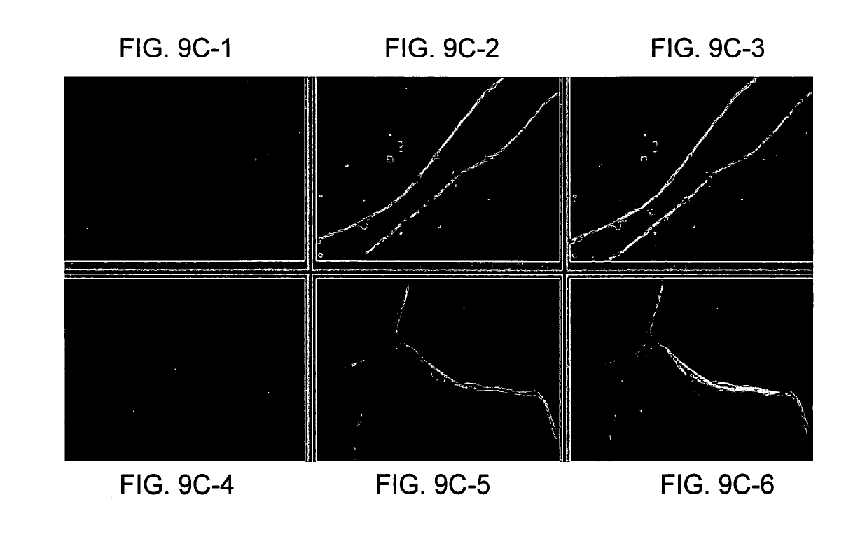

Finally, it was investigated whether the formation of myelin segments requires a minimum of two weeks in culture, or whether less time would be sufficient. Oligodendrocytes were plated on PDL/merosin substrate into dividing medium for two days, the cells were then switched to differentiation medium. The ADORA1 agonist MECA was added to the medium 24 hours later. RGCs were plated onto the oligodendrocytes on the same day that the agonist was administered. Cultures were fixed at 5, 8 and 14 days for immunohistochemistry. The results demonstrate that two weeks was required for the myelin segments to be set up. After five days in co-culture (FIG. 9), there is no significant difference to be observed between control cultures and ADORA1 agonist (MECA) stimulated cultures. After eight days (FIG. 9), myelin was observed covering the whole length of an axon in the stimulated co-cultures, but only after two weeks (FIG. 9) did the segments (internodes) form. This was consistent with the hypothesis that first the oligodendrocyte membrane will form spiral-like loops around the whole length of an axon and that this spiral is subsequently contracted during the process myelin compaction.

### Example 5

### Gene Expression Analysis Comparing Non-Myelinating Oligodendrocytes with ADORA1 Agonist Induced Oligodendrocytes Committed to Myelination

It was demonstrated, by QRT-PCR, that rhoA expression could be used as a secondary marker for an ADORA1 agonist (i.e., MECA) stimulated oligodendrocytes committed to myelination. It was also demonstrated herein that rhoA is consistently down-regulated 24 hours after ADORA1 stimulation compared to control oligodendrocytes. RhoA is a member of the Ras superfamily of small GTPases and functions as a GDP/GTP—regulated binary switch that regulates signal transduction pathways controlling actin cytoskeletal reorganization. RhoA down-regulation through ADORA1 activation has been shown to lead to morphological changes in different cell types like pituicytes (Rosso et al., 2002 Eur. J. Neurosci. 16:2324-2332) and astrocytes. Oligodendrocytes undergo significant morphological changes during myelination, and the switch to the myelinating phenotype in optic nerve myelination cultures is mediated by ADORA1 activation. RhoA expression was characterized in rat optic nerve oligodendrocytes. Expression of rhoA was determined by Quantitative RT-PCR in oligodendrocytes treated with MECA or untreated for various times, as set forth below. Oligodendrocytes were grown in mitogenic media for 3 days and switched to differentiation media for one day before treatment with MECA. RNA was isolated from untreated- or MECA-treated oligodendrocytes at 6 hours, 16 hours, 1 day, 2 days and 3 days. Quantitative RT-PCR was set up as per standard protocol as a single step reaction. The rhoA sequence primer TCC GTC TTT GGT CTT TGC TGA (SEQ ID NO:9), and reagents were obtained from Applied Biosystems.

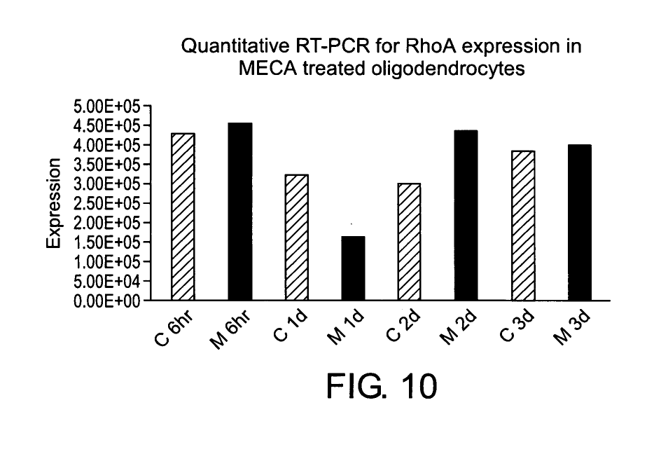

Based on results from three separate experiments (n=3), a two-fold reduction in rhoA expression was observed at day 1 in the MECA-treated oligodendrocytes compared to control (untreated) (FIG. 10). No significant differences were observed at any other time points. From these experiments, it appears that rhoA is a marker for oligodendrocytes committed to myelination. Also, the results demonstrate that treating oligodendrocytes for one day with MECA can provide more significant changes in gene expression profiling studies.

Based on the results obtained with rhoA expression, 24 hours was selected as the first time point in a broader gene expression analysis. Total RNA was isolated from control, and MECA-stimulated oligodendrocytes were harvested 24 hours after the addition of MECA. RNA samples were generated in triplicate, each replicate generated by pooling RNA from three separate oligodendrocyte preps (one prep from 12 litters (10 pups each) of post-natal day 5 SD pups). RNA samples were analyzed by gene expression profiling using the Affymetrix GeneChip™ Rat genome 230 2.0 Array that provides coverage of approximately 30,000 transcripts, i.e., the entire transcribed rat genome. RNA submitted was amplified by Expression Analysis using the MessageAmp II aRNA kit from Ambion to generate sufficient amounts of RNA for hybridization to arrays. A2B5 and cells were plated in 6-well plates and allowed to divide for 48 hours. Approximately 500,000 cells were plated per well. Two micrograms of RNA was subsequently used for analysis.

### Example 6

### Gene Expression Analysis Comparing Myelinating-Competent Versus Myelinating-Incompetent Oligodendrocytes (1 Day ADORA1-Agonist Treatment)

To identify gene expression changes occurring during myelination, total RNA was isolated from control and MECA-stimulated oligodendrocytes 24 hours after the addition of MECA. RNA samples were generated in triplicate, each replicate generated by pooling RNA from three separate oligodendrocyte preps. RNA samples (2 μg/sample) were submitted to Expression Analysis for gene expression profiling using the Affymetrix GeneChip™ Rat genome 230 2.0 Array that provides coverage of approximately 30,000 transcripts. RNA submitted using the MessageAmp II aRNA kit from Ambion to generate sufficient amounts of RNA for hybridization to arrays.

### Example 7

### Differentiation of Transient and Long-Term Changes in Oligodendrocytes

To identify transient versus long-term gene expression changes, control and 3-day ADORA1 agonist (MECA) stimulated optic nerve oligodendrocytes were compared. As described elsewhere herein, 3 days of ADORA1 stimulation were sufficient to obtain myelin segments in the optic nerve myelination system. RNA samples were generated in triplicate, each replicate generated by pooling RNA from two separate oligodendrocyte preps (one prep from 12 litters of post-natal day 4 SD pups). RNA samples were analyzed for gene expression profiling using the Affymetrix GeneChip™ Rat genome 230 2.0 Array that provides coverage of approximately 30,000 transcripts. RNA was amplified using the MessageAmp II aRNA kit from Ambion to generate sufficient amounts of RNA for hybridization to arrays, as previously described. Table 1 shows a summary of differentially regulated genes from comparison of control and 3-day MECA stimulated oligodendrocytes.

TABLE 1
Summary of differentially regulated genes comparing control
with 3-day MECA stimulated ON-oligodendrocytes.
p-value <0.05 3064 genes

| Fold change | Genes |
|---|---|
| >1.5 fold | 1021 |
| >1.2 fold | 2428 |
| >1.1 fold | 2721 |

Gene expression analysis comparing control and 3-day MECA treated ON-oligodendrocytes showed approximately 3,000 genes differentially regulated with a p-value<0.05, i.e., at 95% confidence. Overall, 52% down-regulated changes and 48% up-regulated changes were observed with approximately half of the genes being “known” genes and the other half comprising ESTs or “unknown” genes.

### Example 8

### Gene Expression Analysis Comparing Non-Myelinating Cortical Oligodendrocytes with 1-Day ADORA1 Agonist-Induced (MECA) Cortical Oligodendrocytes Committed to Myelination

As a second filter to determine which genes were commonly regulated between pure optic nerve and enriched cortical oligodendrocytes, gene expression analysis comparing non-myelinating cortical oligodendrocytes with 1-day MECA induced cortical oligodendrocytes committed to myelination was made. Since cortical oligodendrocyte cell preparations yield larger numbers of cells, determining overlap between 1-day MECA treated ON-oligodendrocytes and 1-day MECA treated cortical oligodendrocytes help determine if use of cortical oligodendrocytes for future experiments to test genes of interest is a feasible option. Cortical oligodendrocytes were isolated from 2 litters of newborn SD rat pups. RNA for replicates were isolated from the cells which were plated on 3 separate 6-well tissue culture plates (approximately 500,000 cells per well) with control and treated cells included in separate wells on each plate. The important caveat to keep in mind when comparing gene expression in cortical oligodendrocytes with ON-oligodendrocytes is that the cortical oligodendrocytes are not a pure cell population. Cortical oligodendrocytes comprise approximately 80% oligodendrocytes and about 20% astrocytes, with some neuronal cells. Total RNA was generated in triplicate and hybridized to Affymetrix GeneChip™ Rat 230 2.0 arrays with approximately 30,000 genes. RNA submitted for analysis was amplified using the MessageAmp II aRNA kit from Ambion to generate sufficient amounts of RNA for hybridization to arrays and to keep sample preparation conditions similar to 1-day control and MECA treated ON-oligodendrocytes set forth elsewhere herein. Table 2 provides a summary of differentially regulated genes from comparison of control and 1-day MECA stimulated cortical oligodendrocytes.

TABLE 2
Summary of differentially regulated genes comparing control
with 1-day MECA stimulated cortical oligodendrocytes.
p-value <0.05 2285 genes

| Fold change | Genes |
|---|---|
| >1.5 fold | 252 |
| >1.2 fold | 1425 |
| >1.1 fold | 1969 |

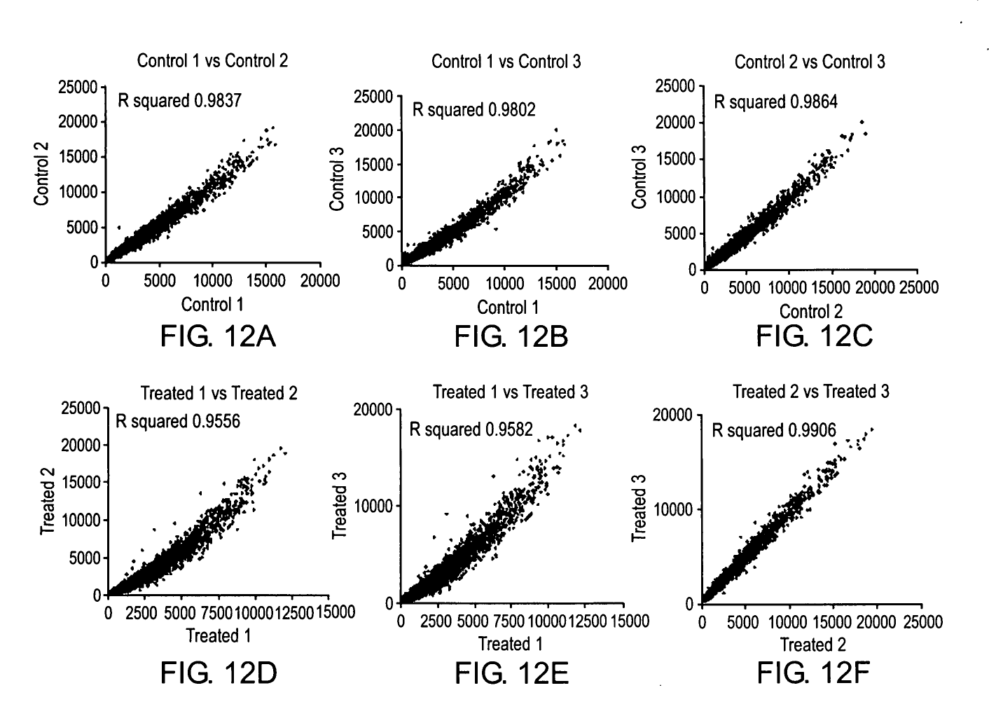

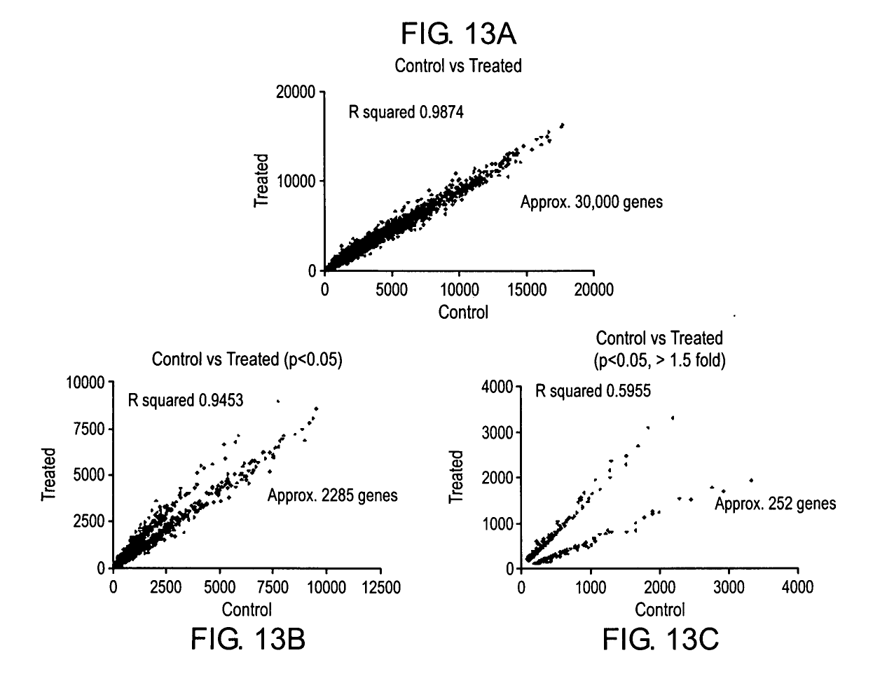

Gene expression analysis comparing control and 1-day MECA-treated cortical oligodendrocytes showed approximately 2,285 genes differentially regulated with a p-value<0.05, i.e. at 95% confidence. Overall, 49% down-regulated changes and 51% up-regulated changes were observed, with approximately half known-genes and half ESTs or unknown genes. As illustrated in FIG. 12, comparison of intensity values within the control and treated replicates shows good reproducibility, as is observed from the correlation coefficient R squared of 0.98 for control replicates and ranging from 0.95 to 0.99 for treated replicates. The slightly lower correlation coefficient for cortical oligodendrocyte replicates compared to the 1-day ON-oligodendrocyte replicates may be attributed to the fact that the cortical oligodendrocyte cultures comprises only approximately 80% oligodendrocytes as mentioned above. Comparison of gene intensities in control versus treated in FIG. 13 shows a correlation coefficient R squared of 0.98, with a slightly lower R squared of 0.94 for genes with p-value<0.05 indicating more changes in expression as is expected.

Differentially regulated genes were compared between two groups of cells. The first group included 1-day MECA-treated ON-oligodendrocytes, and the second group included 1-day MECA-treated cortical oligodendrocytes with a p-value of <0.05 and having a 1.2 fold-change cutoff. The comparison demonstrated an overlap of 414 genes, with 1417 genes that were exclusive to 1-day MECA-treated ON-oligodendrocytes (of 1831 total) and 925 genes (of 1339 total) that were exclusive to 1-day MECA-treated cortical oligodendrocytes. Total numbers of genes corresponding to the two respective groups described above.

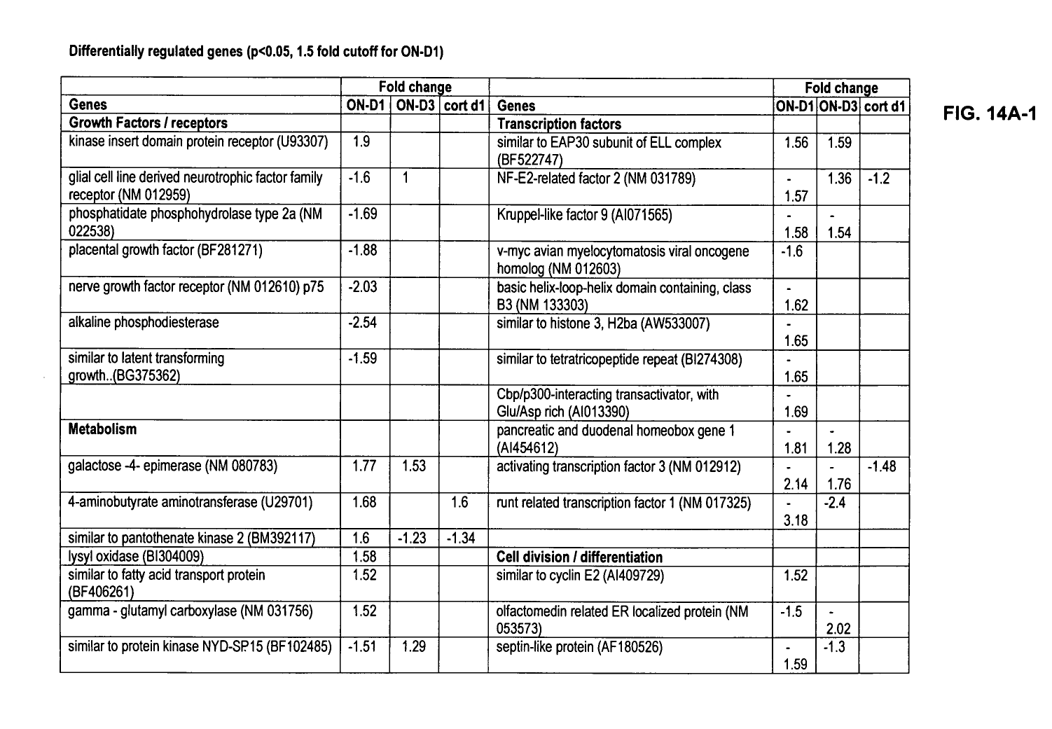

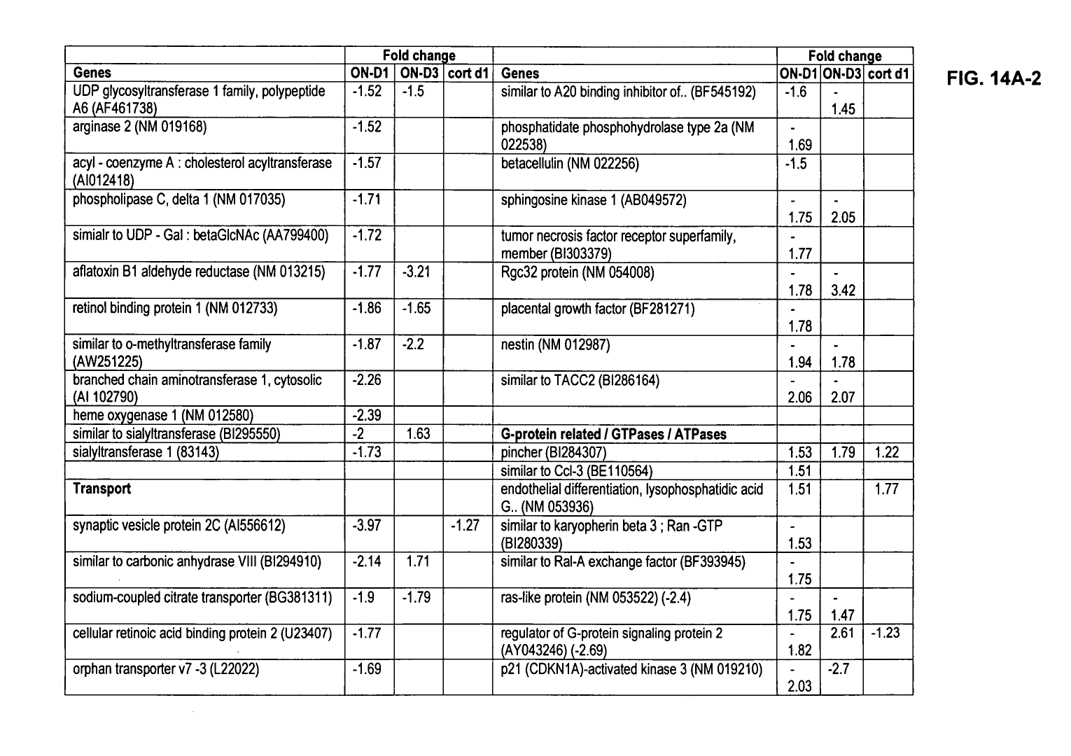

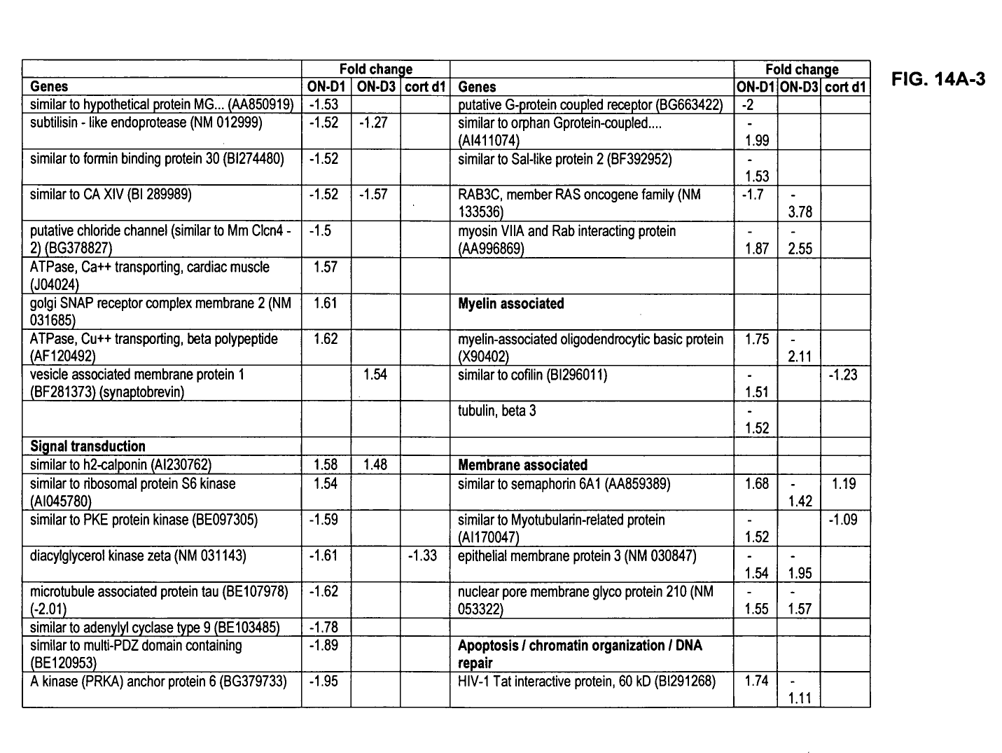

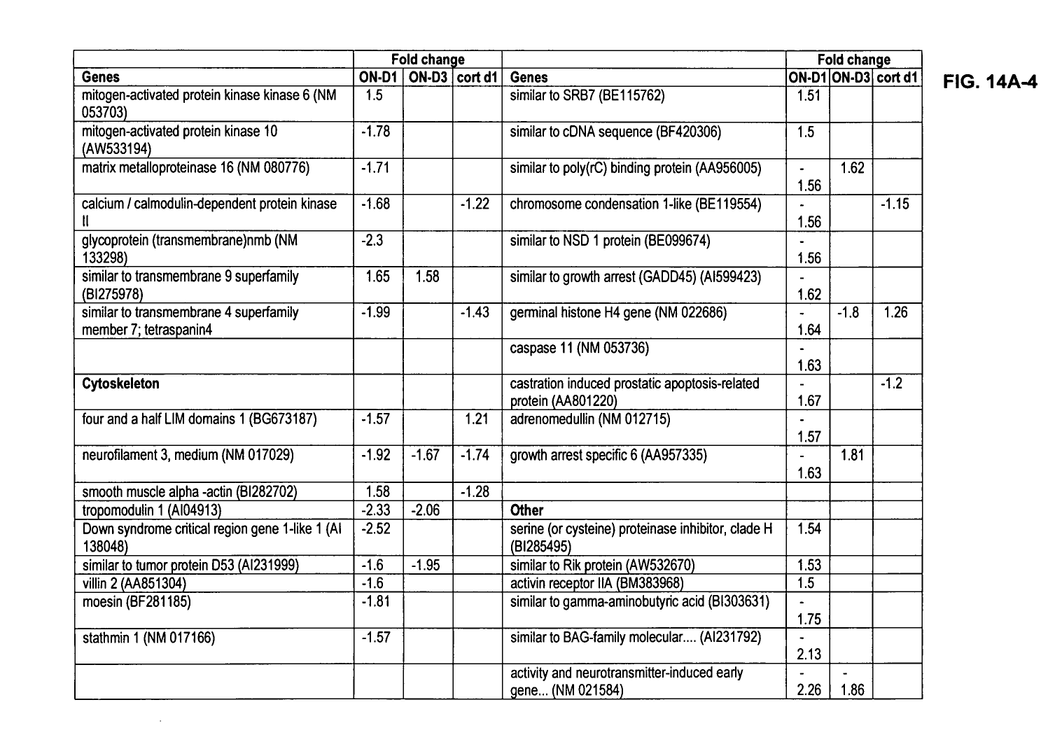

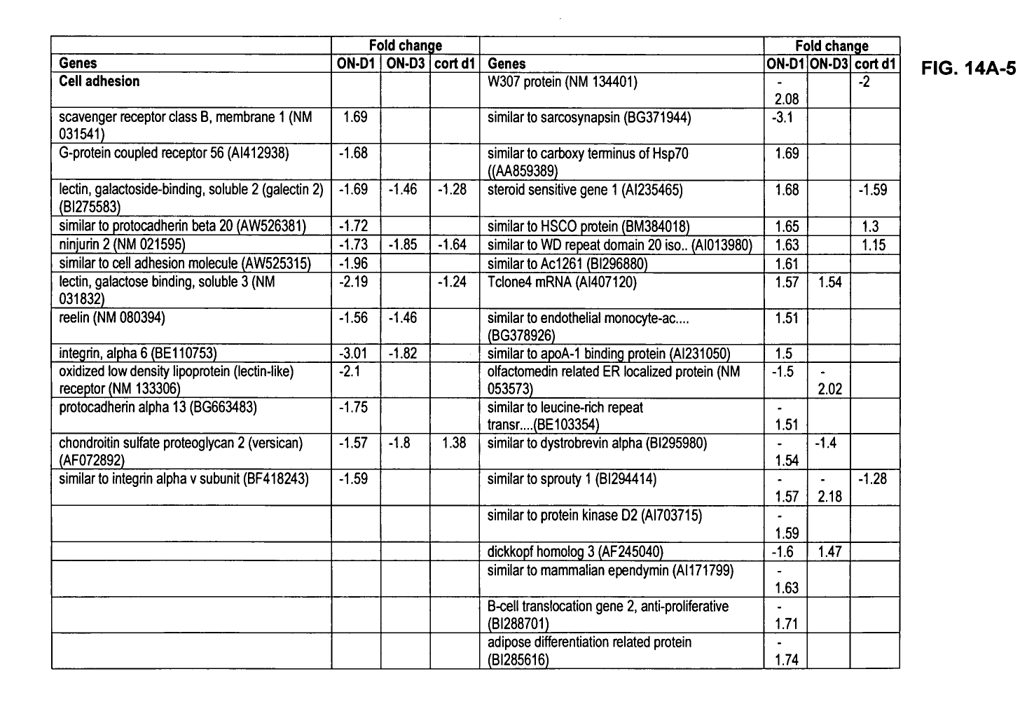

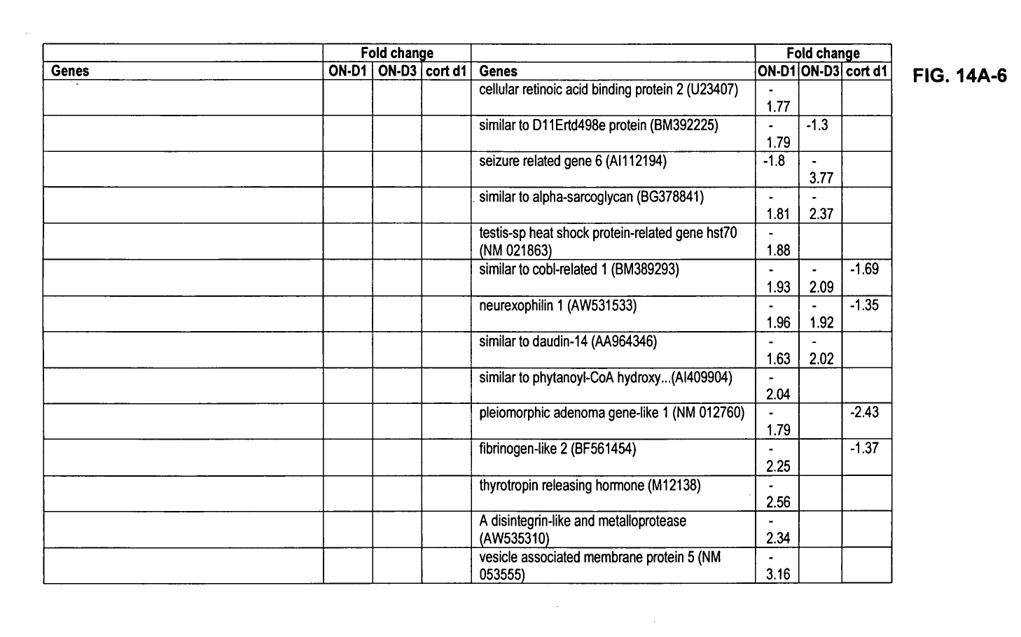

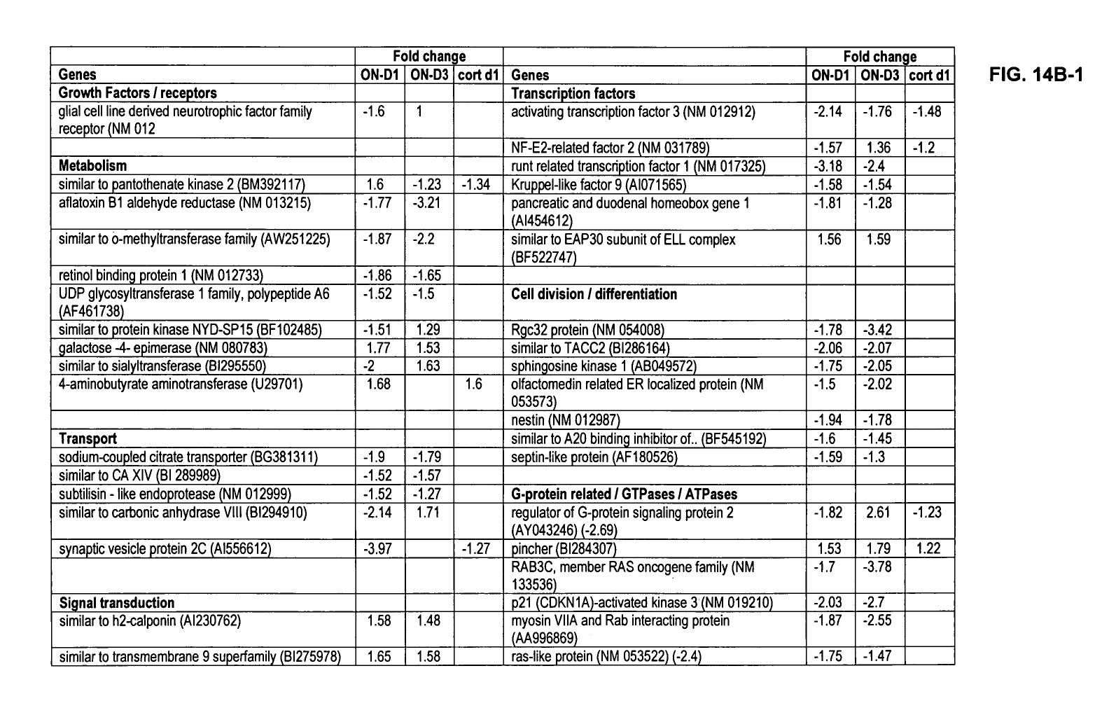

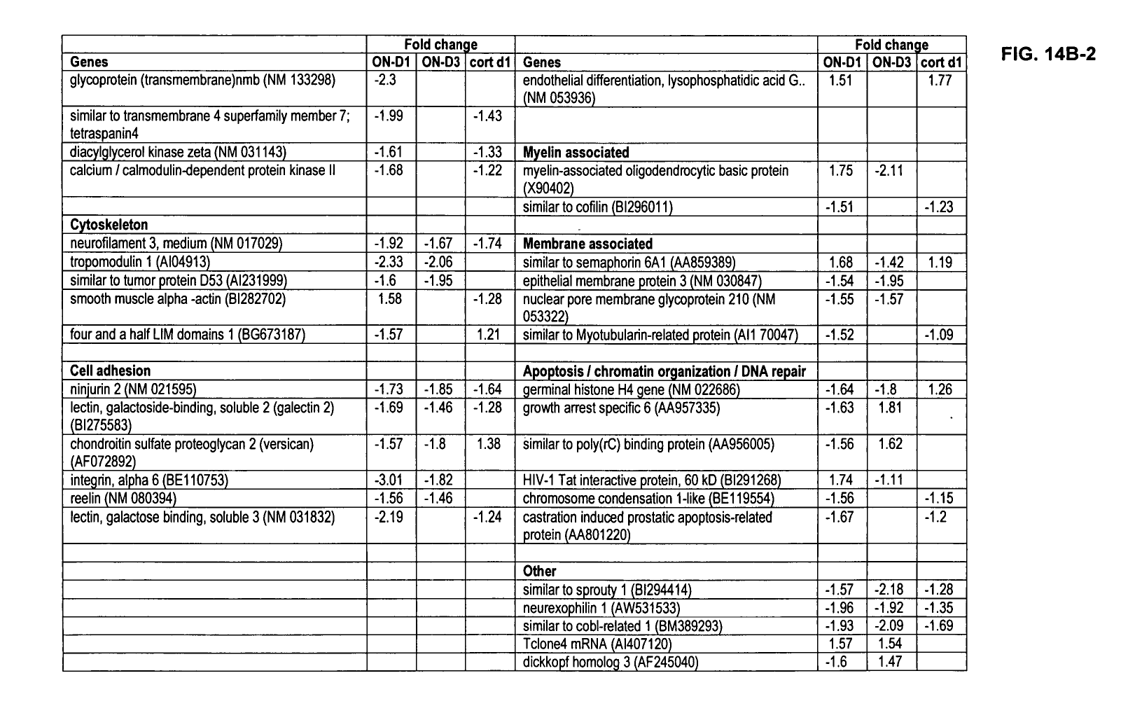

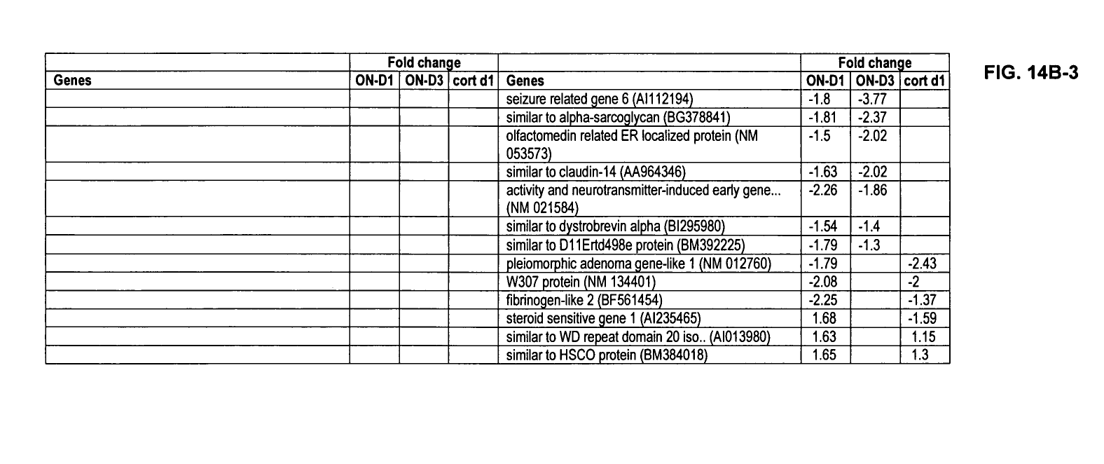

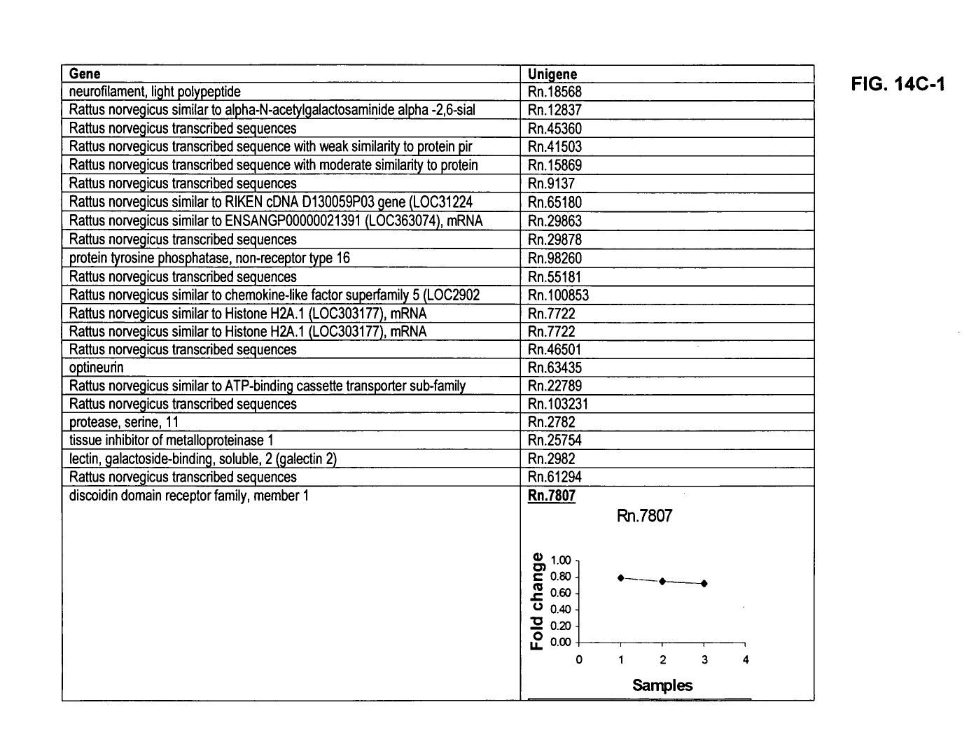

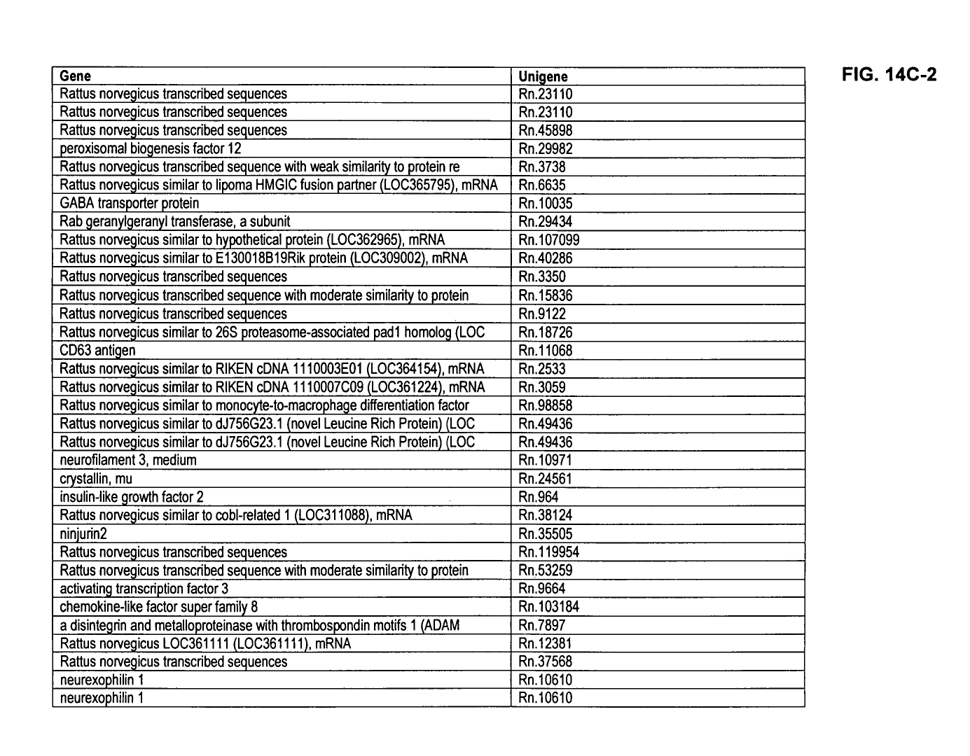

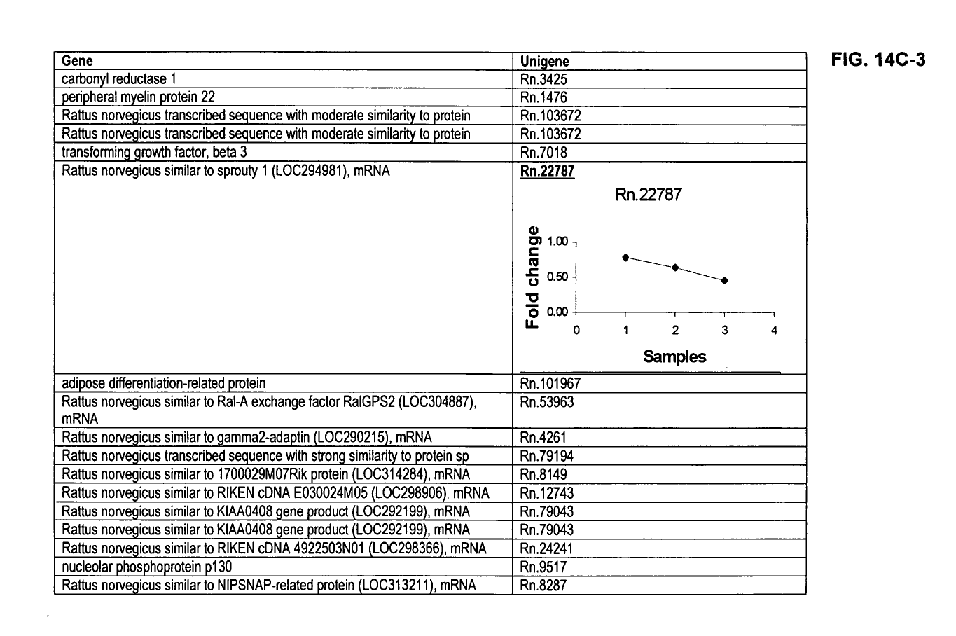

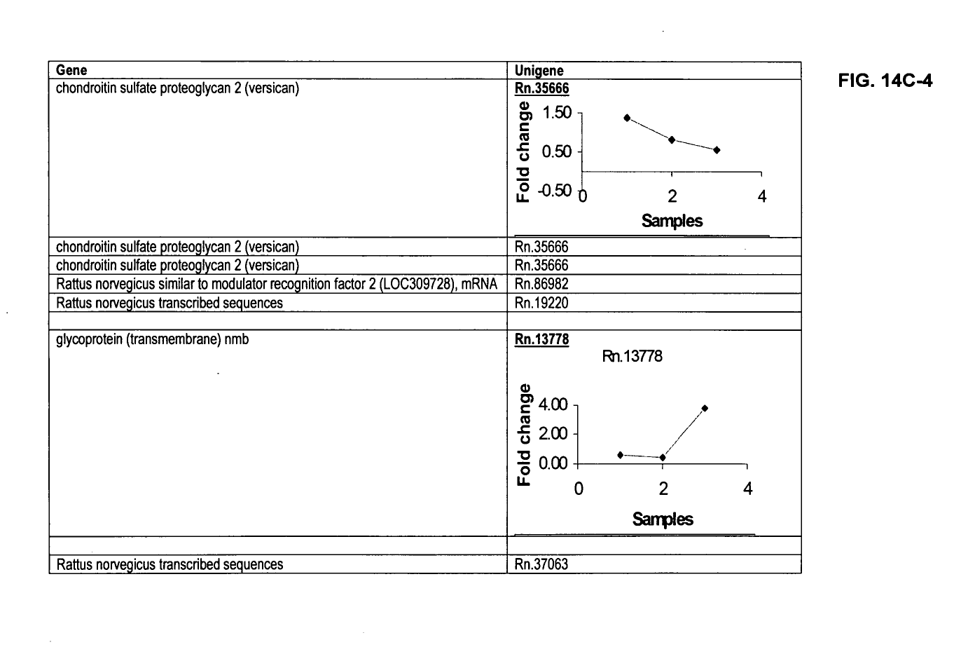

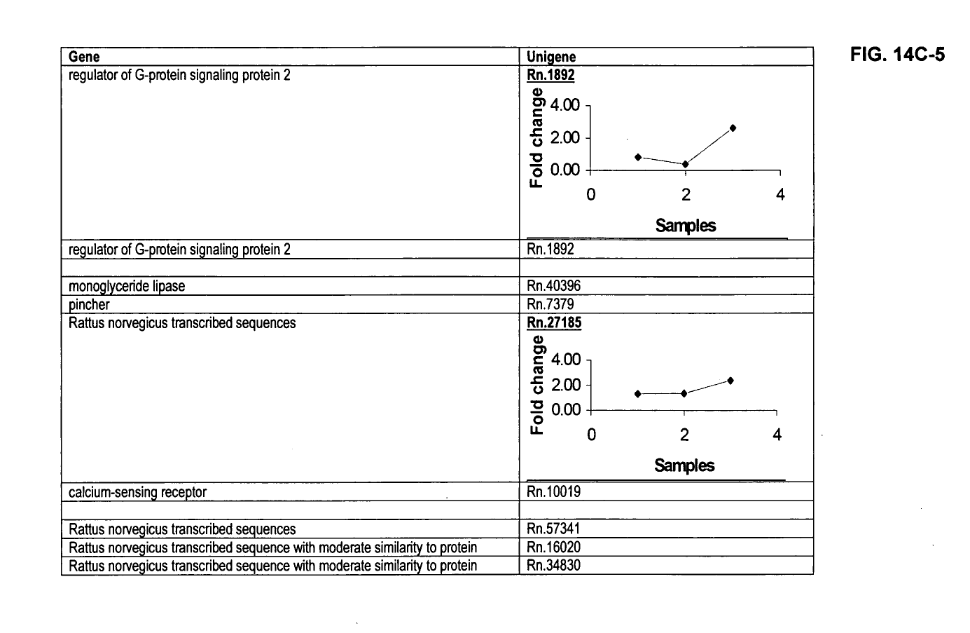

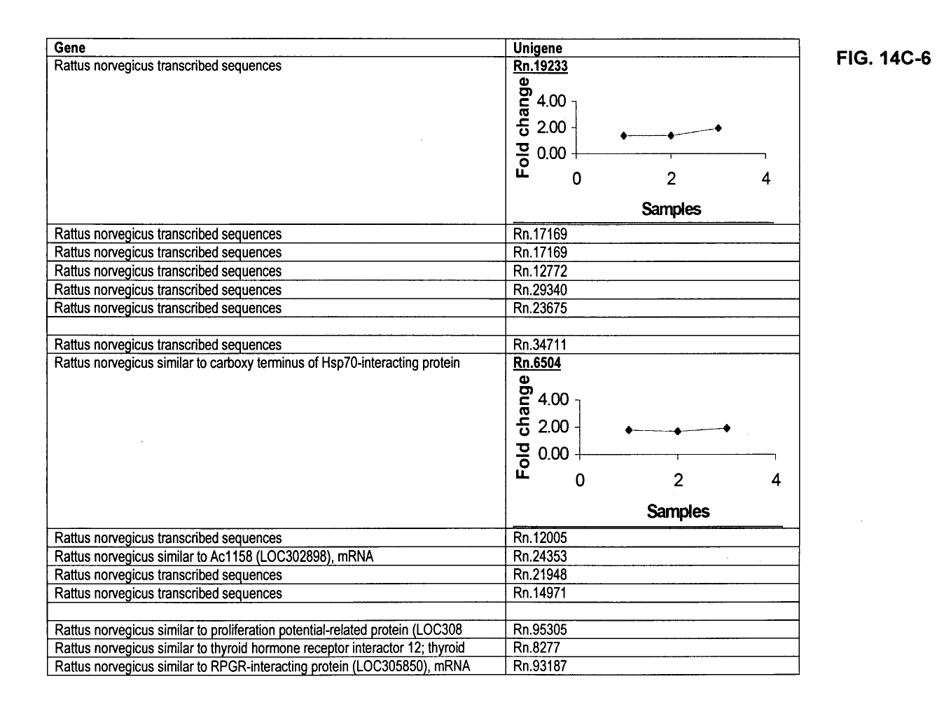

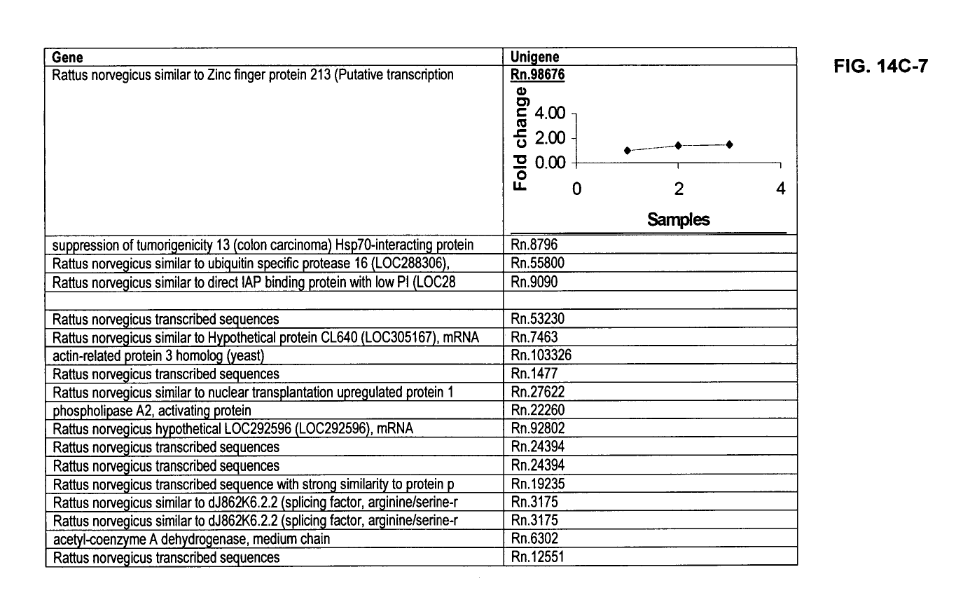

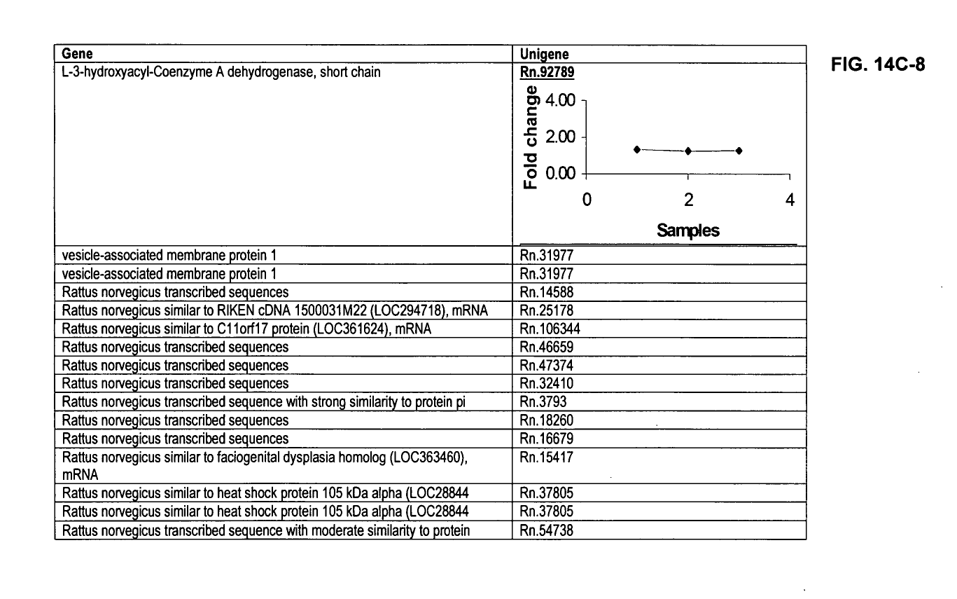

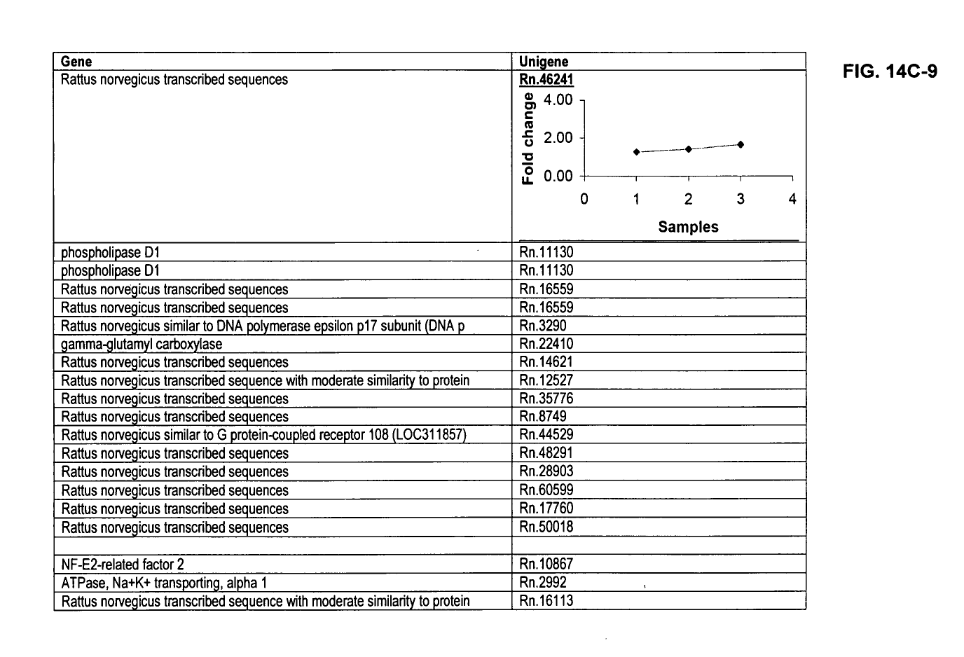

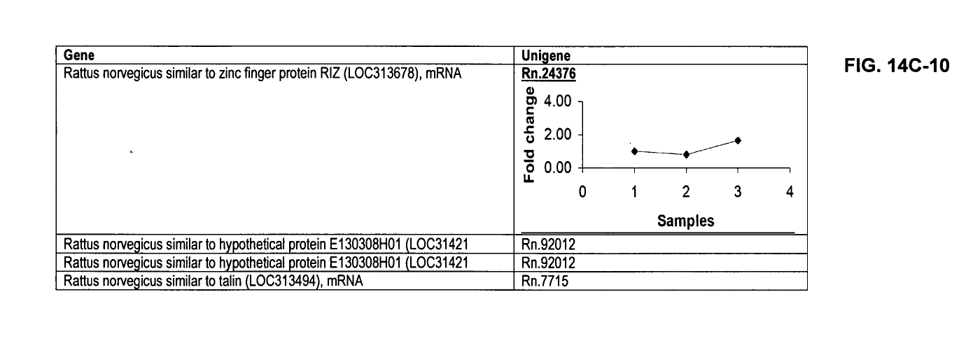

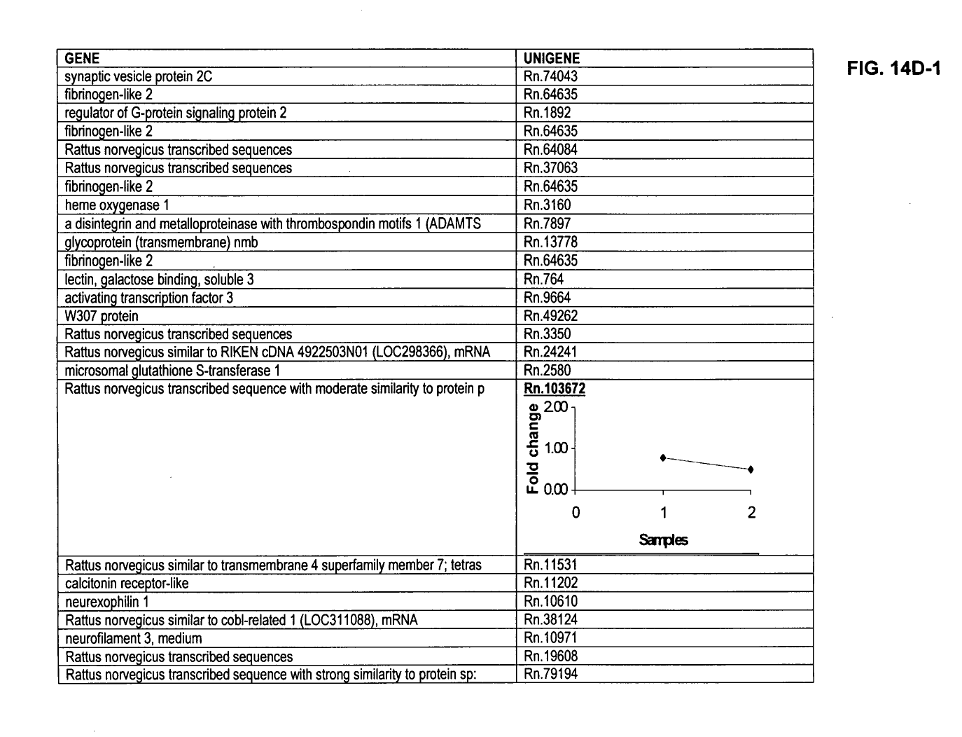

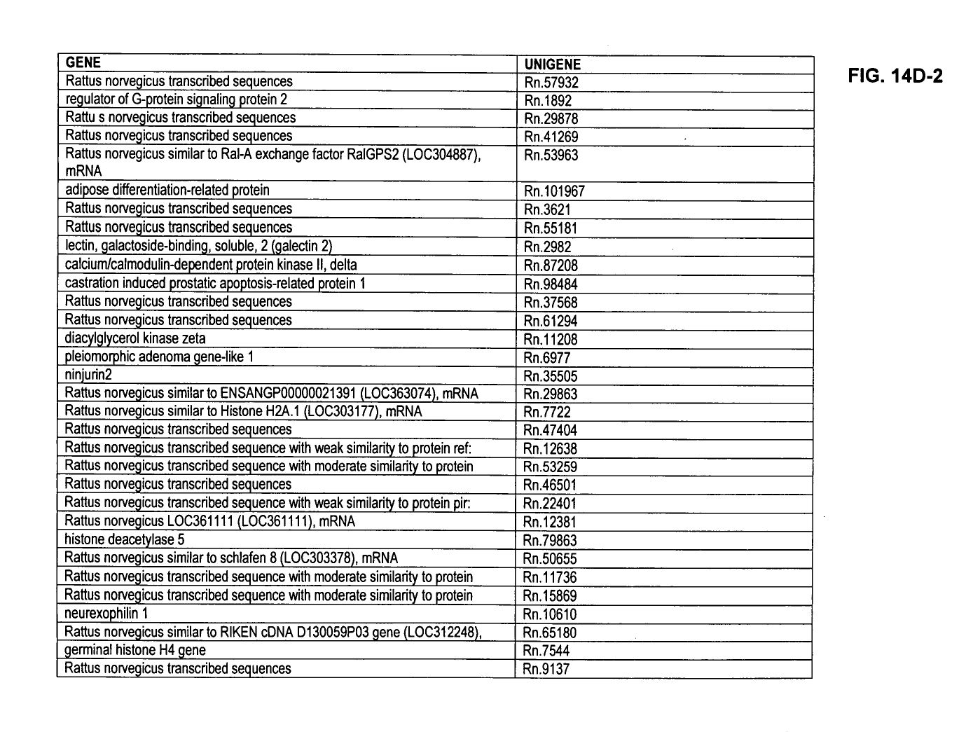

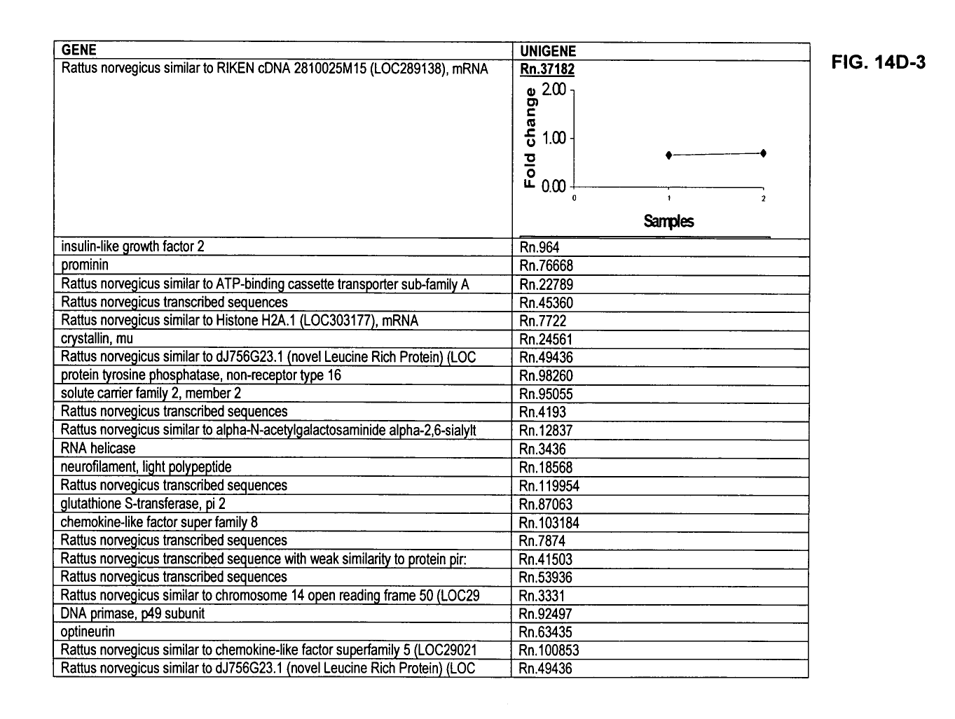

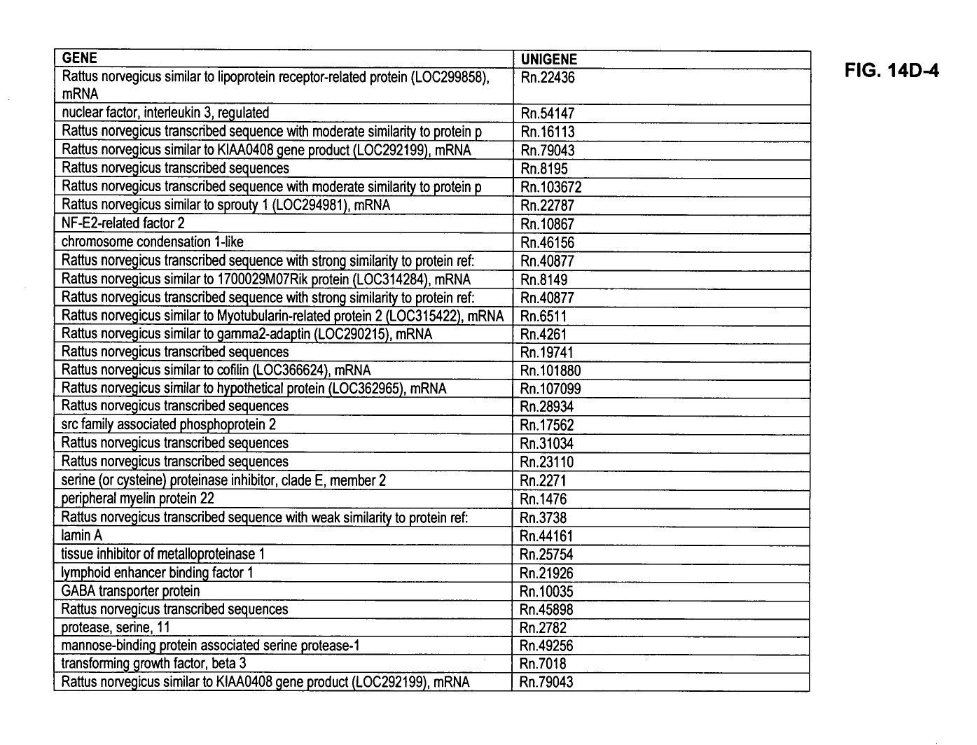

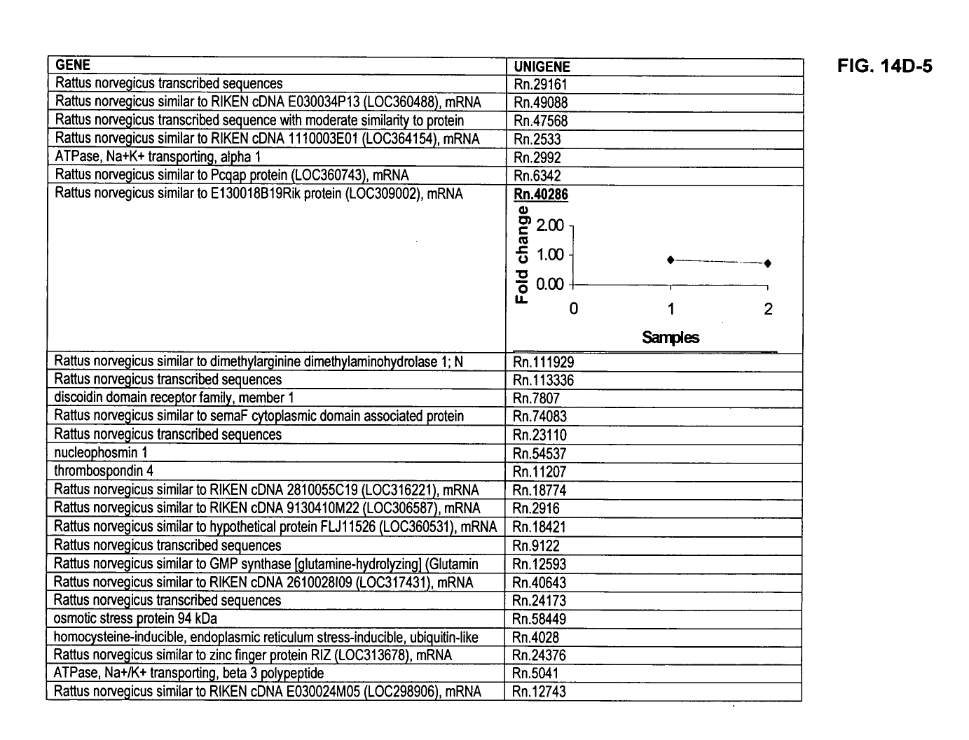

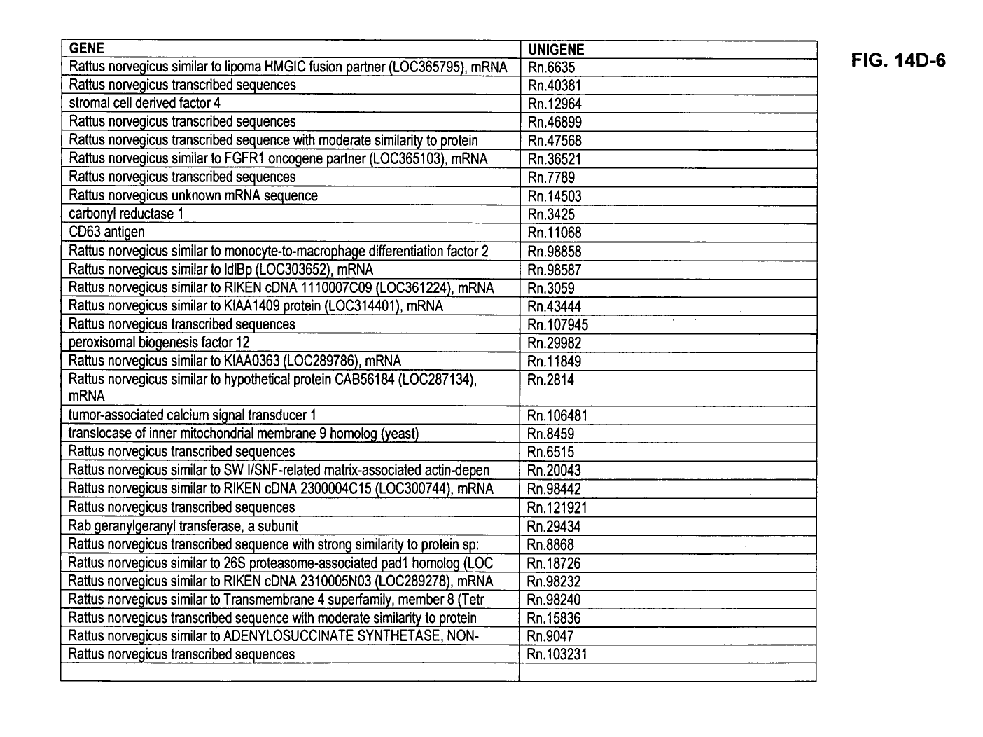

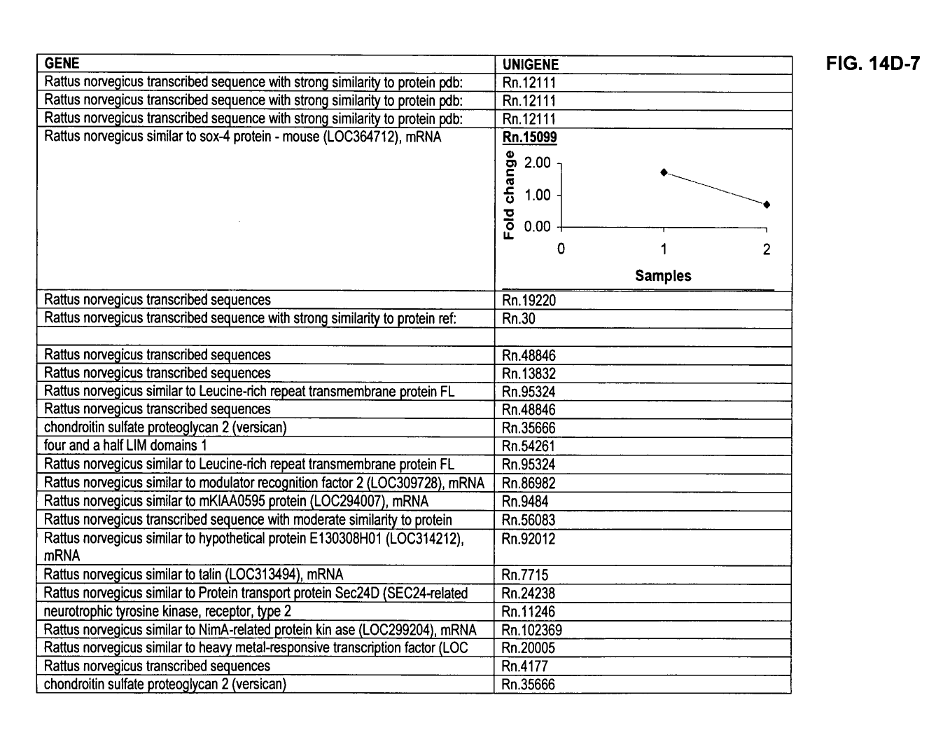

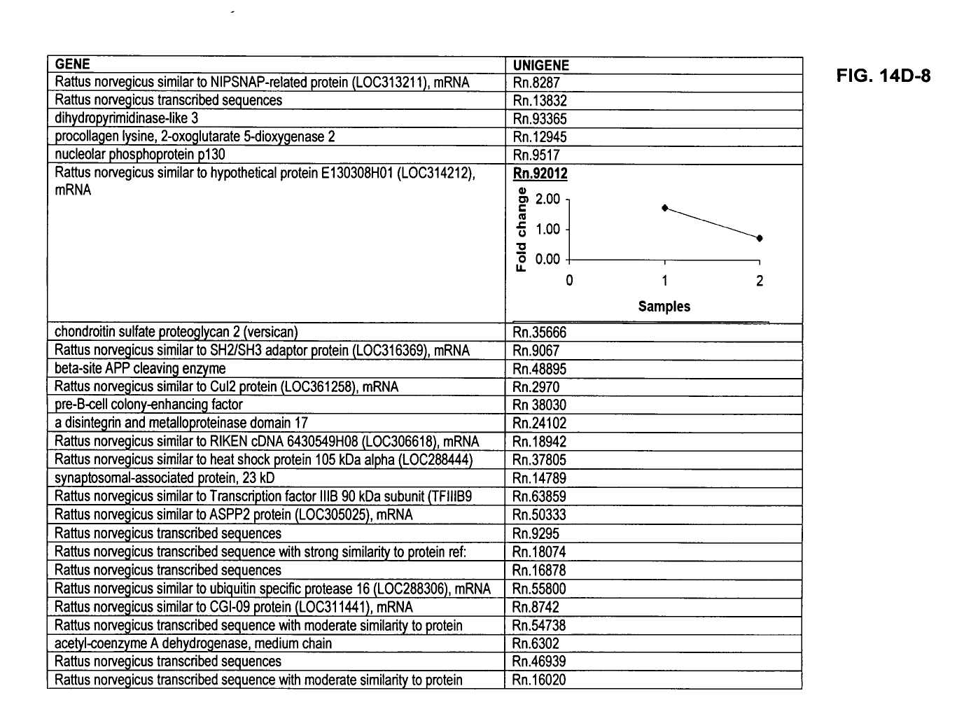

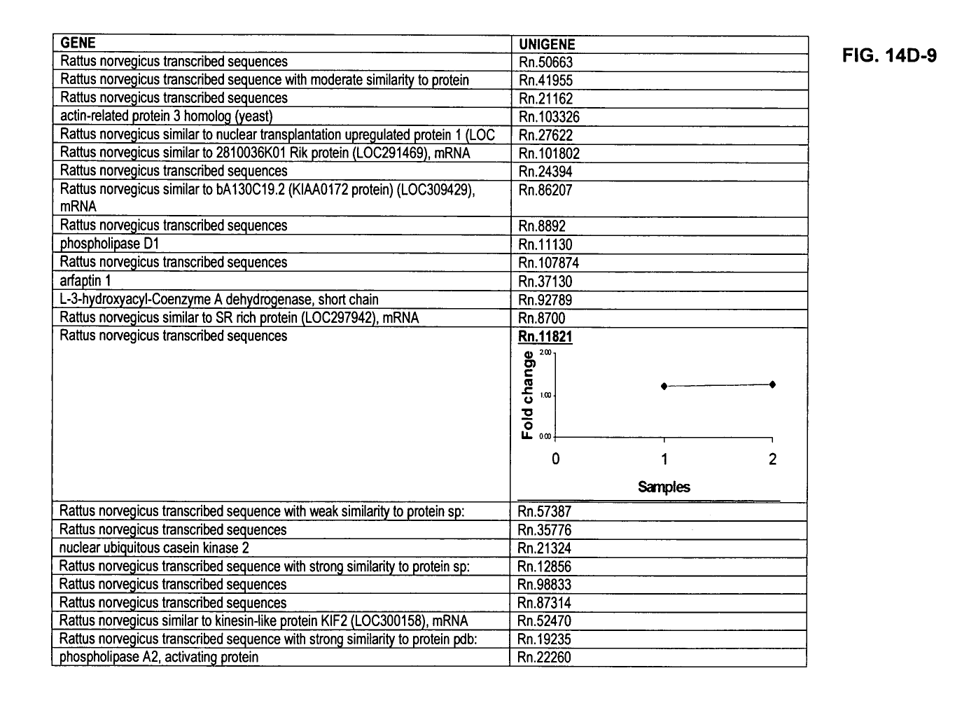

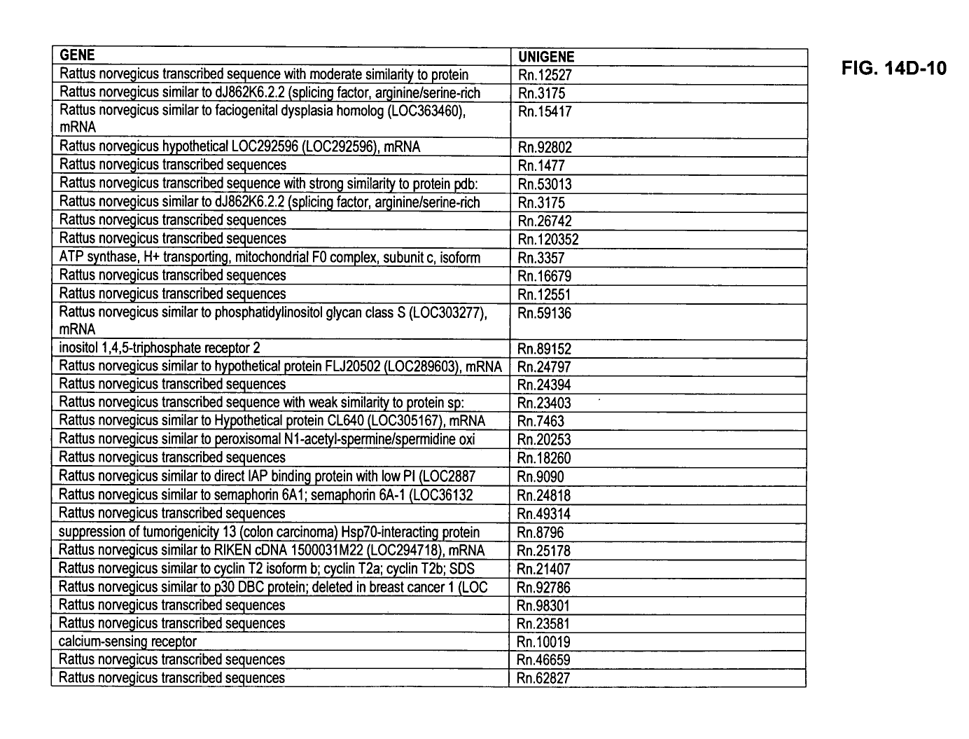

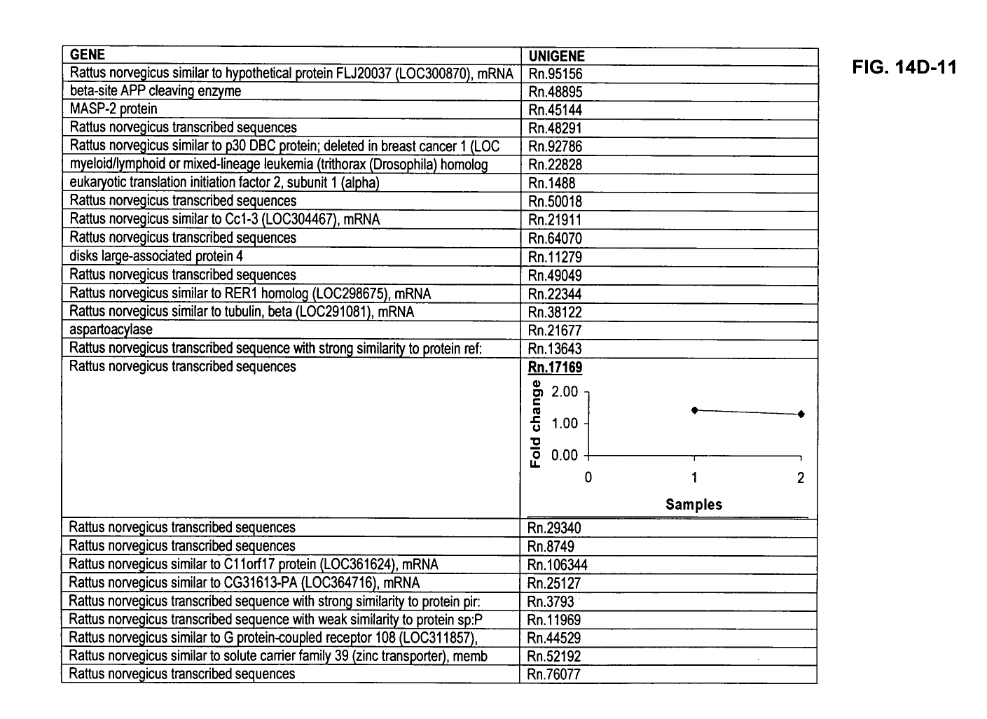

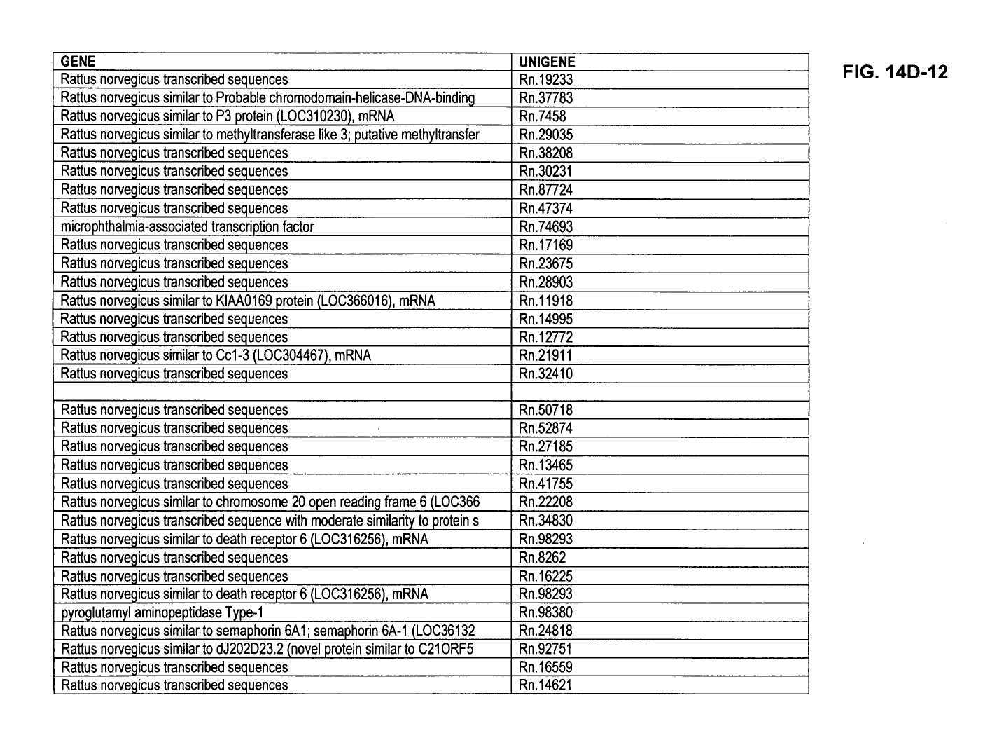

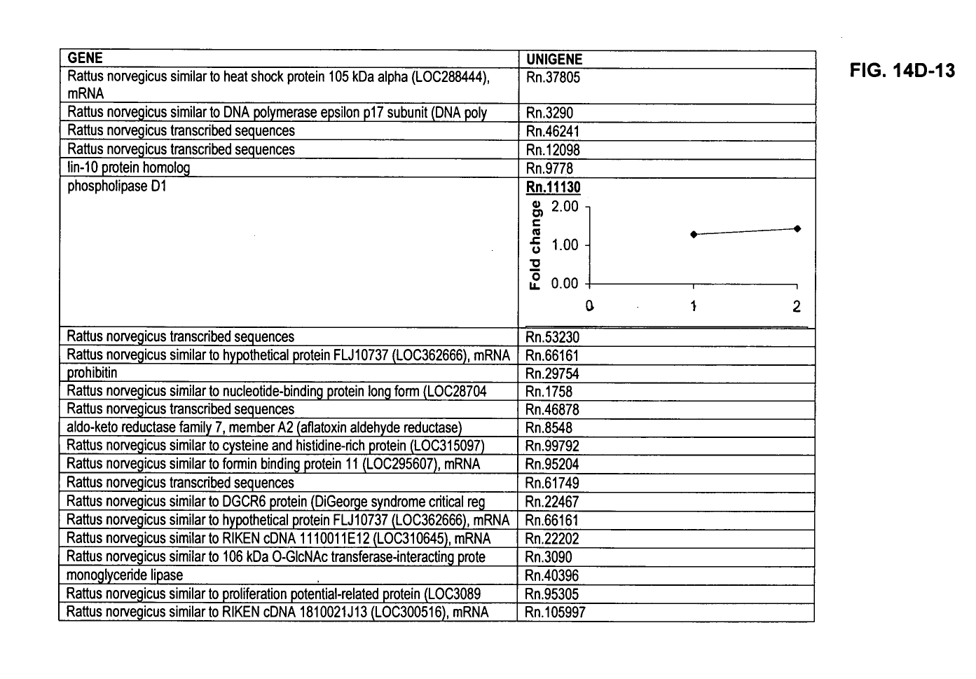

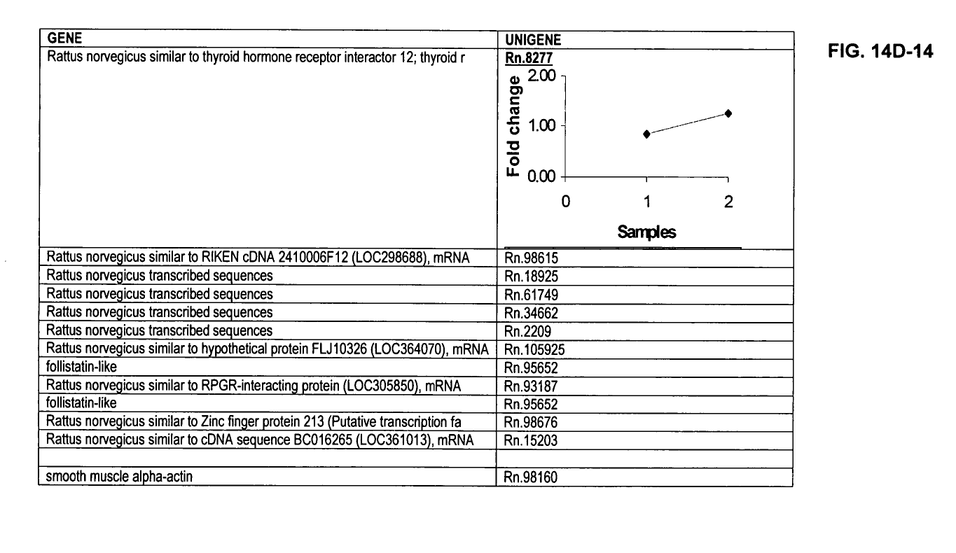

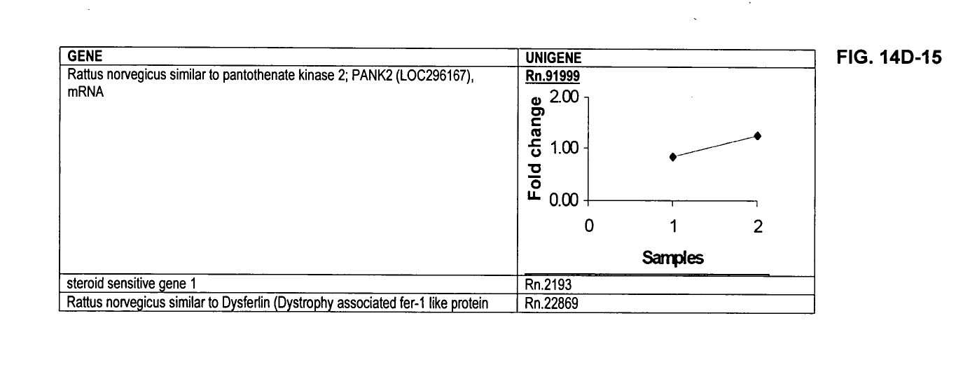

As illustrated in (FIG. 14), comparison of approximately 172 differentially regulated genes from ON-D1 with p-value<0.05 and a 1.5 fold-change cutoff, set forth elsewhere herein, with ON-D3 and cortical d1 oligodendrocytes shows approximately 62 genes differentially regulated in ON-D3, 33 genes differentially regulated in cortical d1 oligodendrocytes and 15 genes differentially regulated in all three samples Genes differentially regulated have been grouped into broad functional classes. In addition to the known genes listed in the table, there are a number of ESTs that show large magnitude of either up or down-regulated changes.

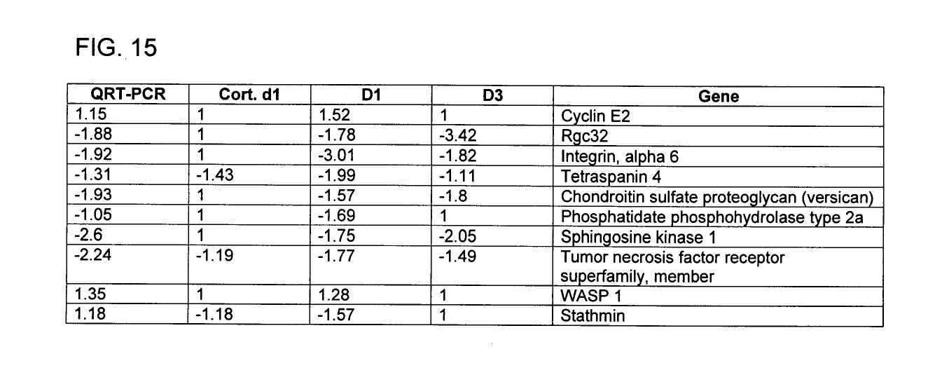

One difficulty that may arise in expression analysis is difficulty in determining expression of genes that were expressed at a low level. QRT-PCR was used to confirm expression of low expressing genes once primers/probes were designed to specific regions of the sequence corresponding to regions to which the Affymetrix probes were designed. Probes for QRT-PCR were designed according to the sequences that are immobilized on the Affymetrix array used in the study. Greater than 90% of differentially regulated genes were confirmed in this way, providing further support for array data (FIG. 15).

Based on the expression studies described herein, 223 genes were identified in common to 1-day ADORA1 agonist MECA-treated myelinating cortical and optic nerve oligodendrocytes and 3-day MECA-treated myelinating optic nerve oligodendrocytes. Within this group of genes are 84 known genes and 89 ESTs. In addition, 45 genes that are in common to only 2 of 3 samples have also been identified as potentially playing a role in myelination. Based on grouping of the genes into functional classes, and after excluding genes involved in general cell processes and genes common to a general polarization process, genes were identified that could be involved in more long-term changes, and were regulated in both cortical and optic nerve oligodendrocytes. Three identified genes include Rgc32, Sphingosine kinase I, and Edg2.

Sphingosine kinase is a regulatory enzyme involved a variety of cellular responses. Its activity can affect inflammation, apoptosis and cell proliferation. Sphingosine kinase can therefore be used as a target to induce remyelination and, additionally, in the treatment of multiple sclerosis and other demyelinating diseases. Modulation can be with an agonist or an antagonist.

### Example 9

### Surrogate Markers for In Vitro Myelination

As described elsewhere herein, myelination in vitro may take two weeks, until segments of myelin are formed. Until now, there had been no described marker that would distinguish a myelinating oligodendrocyte from a non-myelinating one. Therefore, characterizing a screening technique that is both quick and simple was a long-felt need in the field of art. As illustrated elsewhere herein, one of the genes that was regulated and observed in both optic nerve and cortical oligodendrocytes after 3 days and in cortical oligodendrocytes after 24 hours, was the gene for Myelin-Oligodendrocyte Glycoprotein (MOG).

Using an antibody that recognized MOG, it was examined whether MOG protein expression was also regulated in myelinating versus non-myelinating oligodendrocytes. This would provide another marker for myelination in vitro.

Purified rat optic nerve and enriched rat cortical oligodendrocytes were first grown in mitogenic medium and then switched to differentiation medium. Stimulated oligodendrocytes received the ADORA1 agonist, MECA, 24 hours later. Optic nerve oligodendrocytes were assessed 2, 4, and 6 days post MECA treatment, cortical oligodendrocytes, 3, 5, and 7 days post treatment. For the MOG cell surface label, cells were incubated with the anti-MOG antibody (1 μg/ml) for one hour at room temperature in phosphate buffered saline (PBS) containing sodium-pyruvate and glucose (Invitrogen) to ensure survival of cells during this time. Subsequently the cells were fixed, incubated in PBS containing 50% goat serum at RT for 30 minutes and incubated with a secondary antibody (FITC- or Cy3-coupled goat anti-mouse IgG antibody from Jackson Immunoresearch).

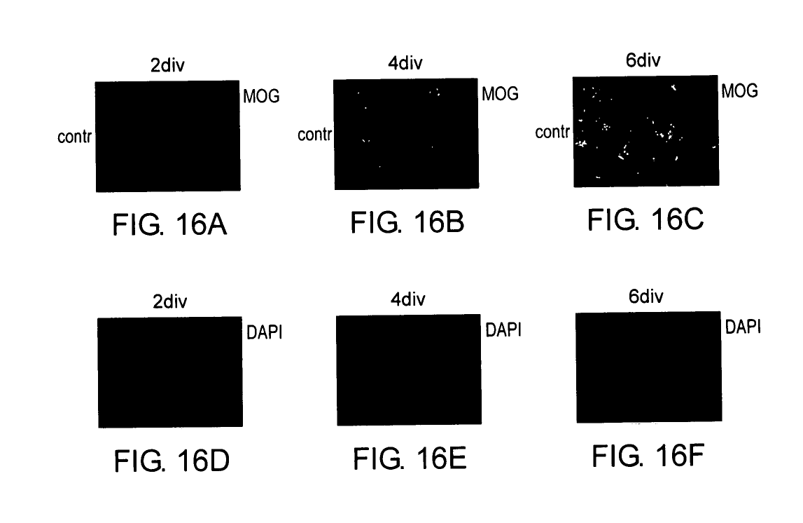

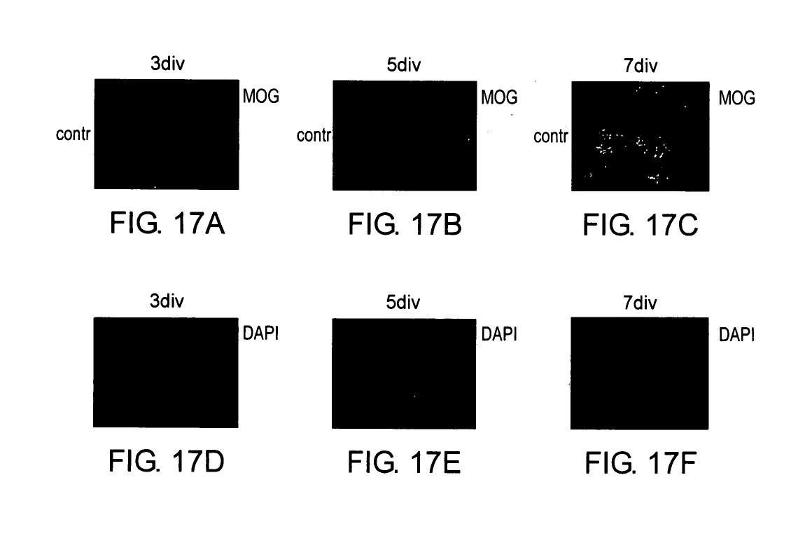

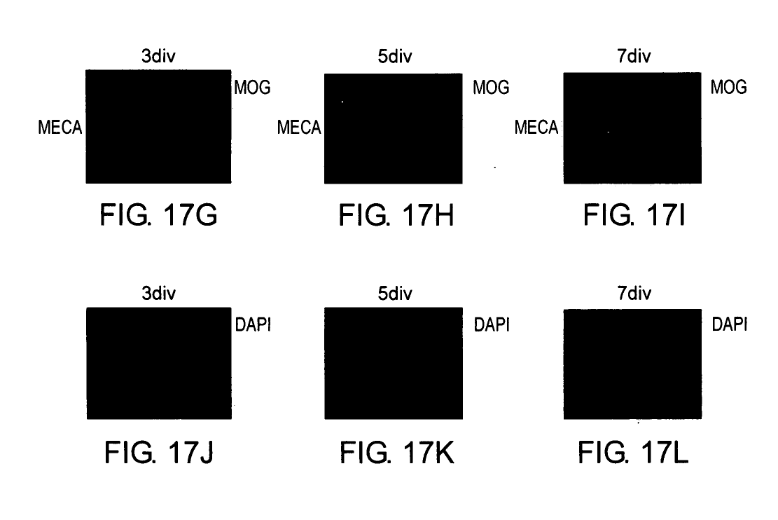

It was found that MOG protein expression on the cell surface of oligodendrocytes is significantly regulated in control, non-myelinating oligodendrocytes versus stimulated myelinating oligodendrocytes after 6 days in the optic nerve (FIG. 16) and after 7 days in the cortical oligodendrocytes (FIG. 17). That is, control oligodendrocytes up-regulated MOG protein expression, whereas the expression of MOG in stimulated oligodendrocytes remained low.

To determine whether the surface expression of MOG would follow the same time course as that observed for the ADORA1 agonist, MECA, in the optic nerve myelination system, oligodendrocyte-enriched cortical cells were plated on PEI-coated plates in mitogenic medium, containing PDGFAA, and NT3 as factors stimulating proliferation. After 3 days, cells were switched to differentiation medium containing CNTF and NT3. 200 μM MECA was added either the same day or 24 hours later and was incubated on the cells for 24 hours, 48 hours, 72 hours, 4 days, and 7 days continuously. The cells were surface-labeled with an antibody to MOG, after the described incubation. For the ADORA1 agonist dose response experiment, MECA was added at concentrations of 200 μM, 100 μM, 50 μM, 25 μM and 12.5 μM, 24 hours after switching to differentiation medium, and was incubated on the cells continuously. On day 7, cells were surface-labeled with an anti-MOG antibody.

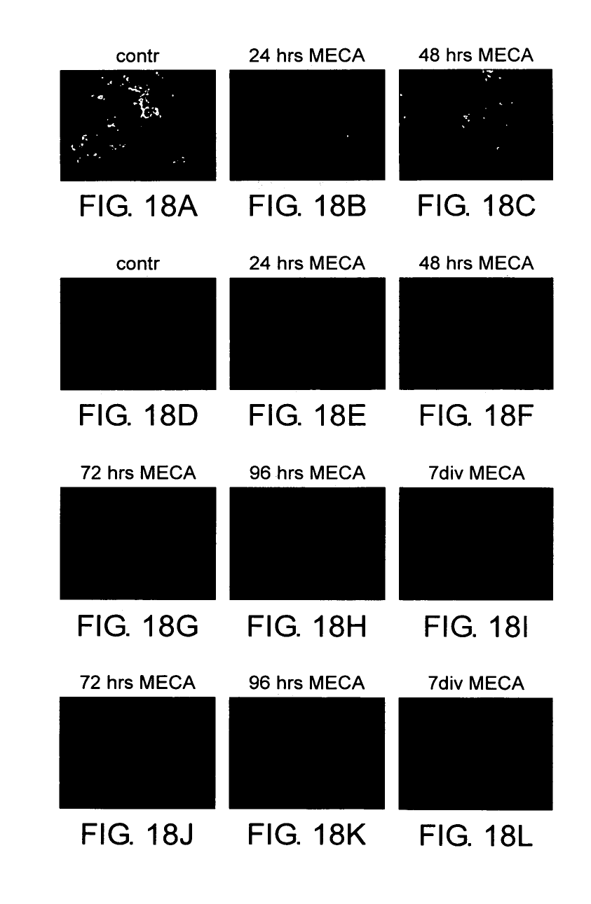

As illustrated in (FIG. 18), MECA was required to be present for at least 72 hours in order to repress MOG surface expression in oligodendrocytes that were switched to myelinating phenotype. This observation correlates with the results regarding optic nerve myelination cultures as set forth elsewhere herein.

To investigate whether the repression of MOG surface expression correlated with the dose needed in order to switch a non-myelinating oligodendrocyte to myelinating phenotype, ADORA1 agonist was examined at concentrations below 100 μM, and was found to be insufficient to repress MOG surface expression on MECA-stimulated myelinating oligodendrocytes. This observation correlates well with the dose-response observed in the optic nerve myelination system, in which doses below 100 μM would not produce myelin segments.

To investigate whether MOG surface expression could be used for RNAi experiments to validate selected genes as a substitute assay for the in vitro myelination system, rat optic nerve oligodendrocytes were grown in mitogenic media for 2 days and switched to differentiation media an hour before transfecting with 500 ng. As described elsewhere herein, multiple siRNA sequences were designed in order to screen for RNAi. Transfections were set up using 1.5 μl of transfection reagent Lipofectamine 2000 (Invitrogen) and 500 ng siRNA in 100 μl of OptiMEM per reaction. The cells were then with these reagents for 20 minutes at room temperature and added to oligodendrocytes in 500 μl differentiation media. Oligodendrocytes were incubated with media plus transfection reagent for 3 hours followed by a medium change from transfection medium to differentiation/growth medium and cells were then incubated for either 24 hours or for 8 days post-transfection. Total RNA was isolated either 24 hours or 8 days post-transfection using an RNA isolation kit (Ambion). A negative control siRNA directed against beta-secretase was also used, as beta-secretase is an enzyme that is not expressed in oligodendrocytes. Expression level reduction was determined by QRT-PCR.

The sequence of the siRNAs used in the surrogate marker studies were obtained from Ambion, and include the following:

| | |
|---|---|
| 5′ to 3′: | |
| GUGCAGACUCGUUGUACAGtt (sense) | (SEQ ID NO:10) |
| CUGUACAACGAGUCUGCACtc (antisense) | (SEQ ID NO:11) |
| 5′ to 3′: | |
| GGAGUUUGAGAUUCUUAUGtt (sense) | (SEQ ID NO:12) |
| CAUAAGAAUCUCAAACUCCtt (antisense) | (SEQ ID NO:13) |

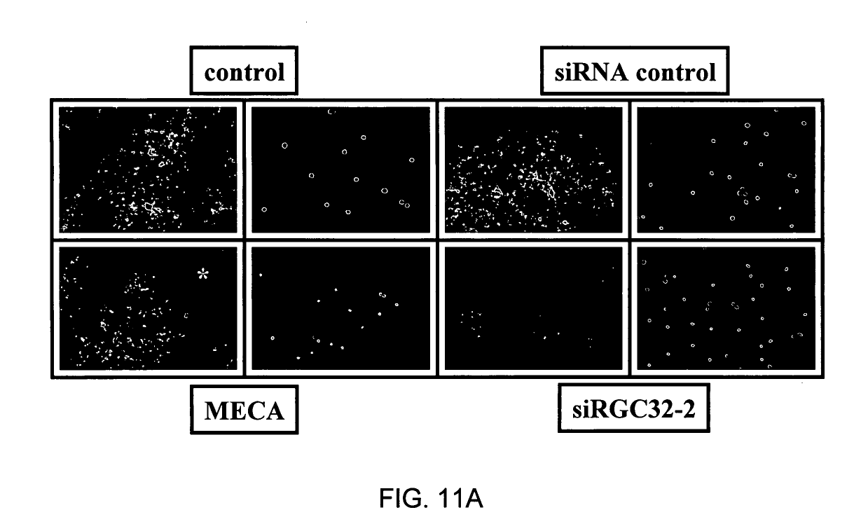

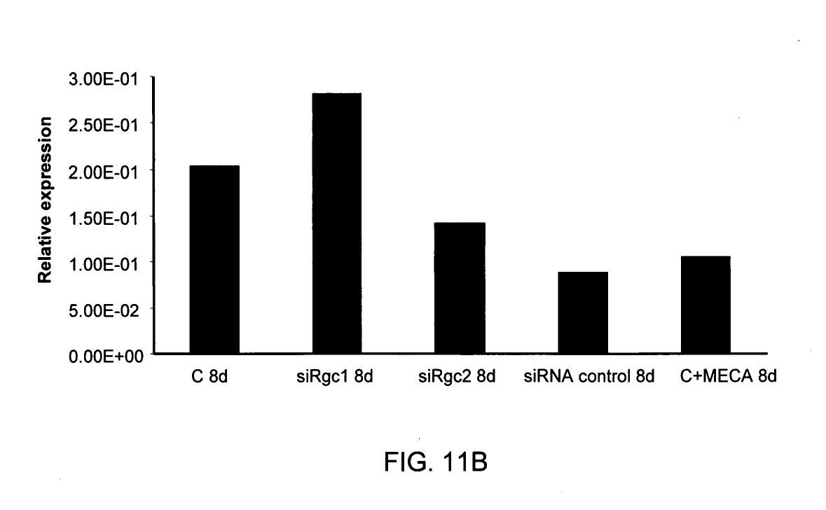

FIG. 11A illustrates MOG surface staining with a MOG-specific antibody that recognizes the extracellular domain of MOG. As shown, MOG expression is reduced when RGC32 is inhibited. RGC32 was another gene that was down-regulated in the optic nerve oligodendrocytes treated with ADORA1 agonist. MOG expression was also analyzed using QRT-PCR, and FIG. 11B demonstrates again that MOG expression is reduced when RGC32 is inhibited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

All references cited above are incorporated herein in their entirety for all purposes.

The present is entitled to priority under 35 U.S.C. § 119(e), to U.S. Provisional Application No. 60/652,477, filed on Feb. 11, 2005, which is hereby incorporated herein by reference in its entirety.

What is claimed is:

1. A method for identifying a gene that is regulated during myelin expression in an oligodendrocyte, said method comprising:
(a) comparing the gene expression profiles of at least two oligodendrocytes, wherein said oligodendrocytes are differentiated in vitro to the state of myelin protein expression, further wherein a first oligodendrocyte is differentiated in the presence of an adenosine receptor agonist, and a second oligodendrocyte is differentiated in the absence of said adenosine receptor agonist;
(b) identifying a molecule, the expression of which molecule is altered in response to adenosine receptor agonist treatment, wherein the altered expression of said molecule is an indication that the oligodendrocyte in which said expression is altered is competent to form compact myelin; and
(c) identifying the gene encoding said molecule.

2. The method of claim 1, wherein said oligodendrocyte is selected from the group consisting of an optic nerve oligodendrocyte, a cortical oligodendrocyte, and a spinal cord oligodendrocyte.

3. The method of claim 1, wherein said oligodendrocytes are essentially pure.

4. The method of claim 3, wherein said oligodendrocytes are at least 95% pure.

5. The method of claim 1, wherein said identifying step comprises at least one of the methods selected from the group consisting of interfering RNA (RNAi), a lentiviral expression system, an adenoviral expression system, a blocking antibody, and a cell permeable inhibitor.

6. The method of claim 1, wherein said molecule is a sphingosine kinase.

7. The method of claim 1, wherein said adenosine receptor agonist is selected from the group consisting of adenosine, MECA, MECA 9-[5(methylcarbamoyl)-β-D-ribofuranosyl]adenosine, CPA (N⁶-cyclopentadyladenosine), CHA (N⁶-cyclohexyladenosine), CCPA (2-chloro-CPA), NECA (N-ethylcarboxamidoadenosine, and XAC (8-[4-[[[[(2-aminoethyl)amino]carbonyl]methyl]oxy]phenyl]-1,3-dipropylxanthine.

8. The method of claim 1, wherein said adenosine receptor agonist is specific for an adenosine A1 receptor.

9. The method of claim 1, wherein said adenosine receptor agonist can bind to at least two different types of adenosine receptors.

10. The method of claim 1, wherein the expression of said molecule is increased.

11. The method of claim 1, wherein the expression of said molecule is decreased.

12. The method of claim 1, wherein said myelination is remyelination.

13. The method of claim 1, wherein said myelination comprises remyelination and new myelination.

14. The method of claim 1, wherein the myelination state of at least one of said oligodendrocytes is confirmed by direct detection of myelination.

15. The method of claim 1, wherein the myelination state of at least one of said oligodendrocytes is confirmed by detection of a surrogate marker for myelination.

16. The method of claim 15, wherein said surrogate marker is myelin oligodendrocyte glycoprotein (MOG).

17. A method for remyelinating at least one neuronal axon in a mammal, said method comprising modulating sphingosine kinase activity in said mammal.

18. A method of stimulating an oligodendrocyte to myelinate, said method comprising modulating sphingosine kinase activity.

19. A method of myelinating an oligodendrocyte, said method comprising modulating sphingosine kinase activity.

20. The method of claim 18 wherein said sphingosine kinase activity is enhanced.

21. The method of claim 20, wherein said sphingosine kinase activity is enhanced with a sphingosine kinase agonist.

22. The method of claim 18, wherein said sphingosine kinase activity is inhibited.

23. The method of claim 22, wherein said sphingosine kinase activity is inhibited with a sphingosine kinase antagonist.

24. A method for treating multiple sclerosis in a mammal, said method comprising administering to said mammal a sphingosine kinase modulator.

25. A method for inhibiting progression of multiple sclerosis in a mammal, said method comprising administering to said mammal a sphingosine kinase modulator.

26. A method for alleviating multiple sclerosis in a mammal, said method comprising administering to said mammal a sphingosine kinase modulator.

27. The method of claim 24 wherein said sphingosine kinase modulator is an agonist.

28. The method of claim 24 wherein said sphingosine kinase modulator is an antagonist.

29. The method of claim 24 wherein said mammal is a human.

30. A cell culture system for identifying a gene that is regulated during formation of myelin in an oligodendrocyte, said culture system comprising:
(a) at least two oligodendrocytes, wherein said oligodendrocytes are differentiated in vitro to a state of myelin protein expression and wherein (i) one of said oligodendrocytes is differentiated in the presence of an adenosine receptor agonist; and (ii) the second of said oligodendrocytes is differentiated in the absence of an adenosine receptor agonist; and
(b) an apparatus for comparing the gene expression profiles of said first and second oligodendrocytes to identify a gene that is regulated during myelin formation.

31. The cell culture system of claim 30, wherein the oligodendrocyte of (a) and the oligodendrocyte of (b) are each independently selected from the group consisting of an optic nerve oligodendrocyte, a cortical oligodendrocyte, and a spinal cord oligodendrocyte.
